(19) United States
(12) Patent Application Publication
(10) Pub. No.: US 2005/0267467 A1
(43) Pub. Date: Dec. 1, 2005

(54) Bipolar conforming electrode catheter and methods for ablation

(76) Inventors: Saurav Paul, Minneapolis, MN (US); Kedar Ravindra Belhe, Minnetonka, MN (US); Chou Thao, Brooklyn Center, MN (US)

Correspondence Address: HEIMBECHER & ASSOCIATES, LLC., 390 UNION BLVD, SUITE 650, LAKEWOOD, CO, 80228-6512, US

(21) Appl. No.: 11/190,724

(22) Filed: Jul. 27, 2005

Related U.S. Application Data
(63) Continuation-in-part of application No. 10/808,919, filed on Mar. 24, 2004.
(63) Continuation-in-part of application No. 10/856,925, filed on May 27, 2004.
(63) Continuation-in-part of application No. 10/856,926, filed on May 27, 2004.
(60) Provisional application No. 60/537,092, filed on Jan. 16, 2004.
(60) Provisional application No. 60/537,092, filed on Jan. 16, 2004.
(60) Provisional application No. 60/537,092, filed on Jan. 16, 2004.

(51) Int. Cl. A61B 18/14

(52) U.S. Cl. 606/41

(57) ABSTRACT

A bipolar conforming electrode catheter has a plurality of flexible filaments or bristles forming a brush electrode for applying therapeutic energy (e.g., RF energy) to target tissue to form spot or continuous linear lesions. Interstitial spaces are defined among the filaments of the brush electrode. The interstitial spaces are adapted to direct conductive or nonconductive fluid, when present, toward the distal ends of the filaments. The brush electrode facilitates electrode-tissue contact in target tissue having flat or contoured surfaces. The flexible filaments may be selectively trimmed to give a desired tip configuration or a desired standoff distance between the tissue and the conductive filaments in the brush electrode. The filaments may be grouped into clusters. The catheter includes a dispersive return electrode for bipolar function.

### CROSS-REFERENCE TO RELATED APPLICATIONS

This application is a continuation-in-part of each of U.S. application Ser. No. 10/808,919, filed 24 Mar. 2004 (the '919 application), now pending; U.S. application Ser. No. 10/856,925, filed 27 May 2004 (the '925 application), now pending; and U.S. application Ser. No. 10/856,926, filed 27 May 2004 (the '926 application), now pending. Each of the '919, '925, and '926 applications claims the benefit of priority of U.S. provisional application No. 60/537,092, filed 16 Jan. 2004 (the '092 application). Each of the '919, '925, '926, and '092 applications is hereby incorporated by reference in its entirety as though fully set forth herein.

### BACKGROUND OF THE INVENTION

a. Field of the Invention

The instant invention is directed toward a conforming electrode catheter and methods for using the conforming electrode catheter for tissue ablation and other forms of treatment. In particular the conforming electrode catheter of the present invention may comprise a brush electrode having a plurality of flexible filaments or bristles for applying therapeutic energy to target tissue for the formation of spot or continuous linear lesions, cauterization, and desiccation.

b. Background Art

Ablation techniques, whereby target tissue is necrotized through coagulation, are performed using catheter devices with electrodes to transfer radio frequency RF energy to tissue. Many benefits may be gained by forming lesions in tissue—for example, control of cardiac arrhythmia or tachycardia, removal of skin diseases, or the treatment of varicose veins—if the depth and location of the lesions being formed can be controlled. In particular, it can be desirable to elevate tissue temperature to around 50° C. until lesions are formed via coagulation necrosis, which changes the electrical properties of the tissue. For example, when sufficiently deep lesions are formed at specific locations in cardiac tissue via coagulation necrosis, undesirable arrhythmia fibrillations or ventricular tachycardia may be lessened or eliminated. “Sufficiently deep” lesions means transmural lesions in some cardiac applications.

Several difficulties may be encountered, however, when attempting to form adequately-deep lesions at specific locations using some existing ablation electrodes. For example, when forming lesions with RF energy, high temperature gradients are often encountered in the vicinity of the electrode. At the edges of some existing electrodes are regions of very high current density, leading to large temperature gradients and hot spots. These “edge effects” may result in the formation of undesirable coagulum and charring of the surface tissue. For example, undesirable coagulum may begin to form when blood reaches around 80° C. for an appreciable length of time, and undesirable tissue charring and desiccation may be seen when tissue reaches around 100° C. for an appreciable length of time. There two types of undesirable coagulum: coagulum that adheres to and damages the medical device; and coagulum blood clots or curds that may enter a patient's bloodstream, possibly resulting in other health problems for the patient. Charring of the surface tissue may also have deleterious effects on a patient.

As the temperature of the electrode is increased, the contact time required to form an adequately-deep lesion decreases, but the likelihood of charring surface tissue and forming undesirable coagulum increases. As the temperature of the electrode is decreased, the contact time required to form an adequately-deep lesion increases, but the likelihood of charring surface tissue and forming undesirable coagulum decreases. It is, therefore, a balancing act trying to ensure that tissue temperatures are adequately high for long enough to create deep lesions, while still preventing or minimizing coagulum formation and/or charring of the surface tissue. Active temperature control may help, but the placement of thermocouples, for example, is tricky and setting the RF generator for a certain temperature becomes an empirical exercise as actual tissue temperatures are generally different from those recorded next to the electrode due to factors such as convection and catheter design.

It is also difficult to ensure adequate tissue contact with existing ablation electrodes. Current techniques for creating continuous linear lesions in endocardial, epicardial, intravascular, or other applications include, for example, dragging a conventional electrode on the tissue, using an array electrode, or using pre-formed electrodes. All of these devices comprise rigid electrodes that do not always conform to the tissue surface, especially when sharp gradients and undulations are present. Consequently, continuous linear lesions are difficult to achieve on trabecular surfaces. When forming lesions in a heart, for example, the beating of the heart further complicates matters, making it difficult to keep adequate contact between the electrode and the tissue for a sufficient length of time to form a desired lesion. With a rigid electrode, it can be quite difficult to maintain sufficient contact pressure until an adequate lesion has been formed. This problem is exacerbated on contoured or trabecular surfaces. If the contact between the electrode and the tissue cannot be properly maintained, a quality lesion is unlikely to be formed.

Catheters incorporating a virtual electrode may address some of these difficulties, but these devices often require high flow rates of conductive fluid (e.g., typically around 70 milliliters per minute) to maintain effective cooling for high-power RF applications. The introduction of a large amount of conductive fluid into a patient's bloodstream may have detrimental effects on the patient.

Thus, there remains a need for an ablation catheter that address these issues with the existing designs and that permits the formation of uniform spot and continuous linear lesions, including transmural lesions, on smooth or contoured surfaces.

The information included in this background section of the specification, including any references cited herein and any description or discussion thereof, is included for technical reference purposes only and is not to be regarded subject matter by which the scope of the invention is to be bound.

### BRIEF SUMMARY OF THE INVENTION

The present invention is a catheter with a new electrode adaptable for a range of surgical applications including ablation, coagulation, cauterization, incision, fulguration, and desiccation. The invention provides a clinician with the ability to form adequately-deep spot or continuous linear lesions in tissue while reducing the formation of undesirable coagulum and charring of the surface tissue. Alternatively, the invention may be used to create therapeutically desired coagulum, for example, to arrest bleeding or to dissipate or occlude varicose veins. This invention also allows a clinician to apply a reasonable amount of therapeutic RF energy, while mitigating electrode-tissue contact problems and/or reducing the amount of conductive fluid (e.g., isotonic saline) possibly entering a patient's bloodstream during the procedure.

In one form, the present invention is a bipolar electrode catheter comprising a flexible sheath with a distal end and a conforming electrode adapted to apply therapeutic energy to target tissue. The conforming electrode has an embedded portion and an exposed portion. The embedded portion extends into the distal end of the flexible sheath. The exposed portion has a distal end that forms a working surface. The exposed portion also extends from the distal end of the flexible sheath. The bipolar electrode catheter also comprises a conductor electrically coupled with the conforming electrode and adapted to carry therapeutic energy from an energy source to the conforming electrode. The bipolar electrode catheter further comprises a return electrode positioned about the flexible sheath proximal to the conforming electrode and a return lead electrically coupled with the return electrode.

The present invention may also take the form of a bipolar surgical device for transferring therapeutic energy to tissue. The bipolar surgical device comprises a flexible sheath having a distal end and defining a fluid lumen and a brush electrode through which the therapeutic energy is applied to target tissue. The brush electrode is mounted at the distal end of the flexible sheath. The brush electrode includes an embedded portion and an exposed portion. The embedded portion is in fluid communication with the fluid lumen. The exposed portion has a distal end forming a working surface. The exposed portion also extends from the distal end of the flexible sheath. The bipolar surgical device also comprises a conductor electrically coupled with the brush electrode and adapted to carry the therapeutic energy from an energy source to the brush electrode. The bipolar surgical device also has outer sheath surrounding the flexible sheath and defining an outer lumen. The distal end of the flexible sheath protrudes distally from a distal end of the outer sheath. The flexible sheath is moveably mounted within and adapted to slide longitudinally with respect to the outer lumen to create an adjustable separation distance between the distal end of the outer sheath and the distal end of the flexible sheath. The bipolar surgical device further comprises a return electrode positioned about an outer surface of the outer sheath and a return lead electrically coupled with the return electrode.

In an alternate form, the invention is directed to a method for treating varicose veins. The method comprises first inserting a bipolar electrode catheter into a lumen of a varicose vein. The bipolar electrode catheter is pulled in a retrograde direction within the lumen of the varicose vein. Contact is maintained between an interior wall of the varicose vein and an annular dispersive return electrode positioned about the bipolar electrode catheter proximal to a distal end of the bipolar electrode catheter. A brush electrode mounted at the distal end of the bipolar electrode catheter is dragged along a length of the interior wall of the varicose vein while pulling the bipolar electrode catheter. The brush electrode is energized with therapeutic energy from an energy source while dragging the brush electrode. The therapeutic energy is transferred from the brush electrode to the interior wall of the varicose vein to occlude the varicose vein. The therapeutic energy is returned from the varicose vein to the bipolar electrode catheter at the annular dispersive return electrode.

Other features, details, utilities, and advantages of the present invention will be apparent from the following more particular written description of various embodiments of the invention as further illustrated in the accompanying drawings and defined in the appended claims.

### BRIEF DESCRIPTION OF THE DRAWINGS

FIG. 1 is an isometric view of one embodiment of a catheter having a brush electrode according to the present invention, and depicts the filaments comprising the brush electrode extending from a distal end of an outer sheath.

FIG. 2 is an enlarged view of the distal end of the catheter of FIG. 1A.

FIG. 3 is similar to FIG. 2, but depicts an alternative embodiment where the brush electrode is secured at the distal end of the catheter by at least one suture that is covered by a section of shrink tube.

FIG. 4 is similar to FIG. 3, but a portion of the shrink tube has been removed to reveal two sutures attaching the brush electrode to the shaft of the catheter.

FIG. 5 is an isometric, cross-sectional view of the catheter depicted in FIG. 3 and 4, taken along line 5-5 of FIG. 3, revealing a primary conductor making electrical contact with a bundle of filaments comprising the brush electrode, and depicting a secondary lead (e.g., for a thermocouple) extending adjacent to the primary conductor and becoming embedded within the brush filaments.

FIGS. 6 and 7 depict steps that may be used to form the brush electrode depicted in, for example, FIG. 5.

FIG. 8 is similar to FIG. 5, but is a cross-sectional view of an alternative embodiment of the brush electrode, wherein conductive filaments are interspersed among relatively longer nonconductive filaments.

FIG. 9 is a cross-sectional view taken along line 9-9 of FIG. 8.

FIG. 10 is an enlarged view of the encircled region of FIG. 8.

FIGS. 11-14 depict alternative shapes of the bundle of filaments at the tip of the brush electrode.

FIG. 15 depicts an alternative embodiment of the filaments of the brush electrode, wherein the individual filaments gradually taper toward their distal ends.

FIG. 16 depicts an alternative embodiment of the filaments of the brush electrode, wherein the individual filaments have nonconductive tips at their distal ends creating a stand-off distance.

FIG. 17 is a fragmentary, isometric view of an embodiment of the shaft of the catheter having a concentric ring of sub-channels or tubes around a main or central channel through which the filaments extend.

FIG. 18 is a fragmentary, isometric view of an embodiment wherein a porous inner sheath surrounds the filaments of the brush electrode adjacent to the exposed portion of the brush electrode.

FIG. 19 is a fragmentary, isometric view of an embodiment wherein a threaded inner sheath, having a spiral or helical ridge on its outer surface, surrounds the filaments of the brush electrode adjacent to the exposed portion of the brush electrode.

FIG. 20 is a fragmentary view of a section of the threaded inner sheath depicted in FIG. 19, surrounded by the outer sheath shown in phantom and cross section to create a helical flow channel between the threaded inner sheath and the catheter shaft.

FIG. 21 is a fragmentary, isometric view of an embodiment wherein a grooved sheath, having a plurality of longitudinally-extending grooves or cuts on its outer surface, surrounds the filaments of the brush electrode adjacent to the exposed portion of the brush electrode.

FIG. 22 is a fragmentary, elevation view of a section of the grooved sheath depicted in FIG. 21, surrounded by the outer sheath (shown in cross section) to create a plurality of longitudinally-extending flow channels between the grooved sheath and the catheter shaft.

FIG. 23 is a fragmentary, cross-sectional view taken along line 23-23 of FIG. 21, with the catheter shaft shown in phantom and with the longitudinally-extending flow channels clearly visible.

FIG. 24 is similar to FIG. 5, but depicts an isometric, cross-sectional view of the shaft of a catheter, wherein the primary conductor makes electrical contact with the filaments via an energy transfer coil or spring surrounding at least the embedded portion of the brush electrode.

FIG. 25 is similar to FIGS. 5 and 24, but depicts an isometric, cross-sectional view of the shaft of a catheter, wherein the primary conductor makes electrical contact with the filaments via an energy transfer mesh or fabric surrounding at least the embedded portion of the brush electrode.

FIG. 26 is a cross-sectional view of a first embodiment of a shielded-tip brush electrode, wherein an uninsulated portion of the primary conductor is looped around the outer surface of the brush electrode.

FIG. 27 is similar to FIG. 26, but depicts a second embodiment of a shielded-tip brush electrode.

FIGS. 28-35 depict different cross-sectional configurations for brush electrodes according to the present invention.

FIG. 36 is a cross-sectional view of a brush electrode wherein some of the filaments comprise hollow or porous members.

FIG. 37 is a cross-sectional view of a brush electrode having devices (e.g., a thermocouple or other temperature sensor, a pressure sensor, or an ultrasound sensor) embedded among the filaments.

FIG. 38 is an isometric view of a catheter having a brush electrode according to the present invention forming a spot or point lesion on a section of tissue.

FIG. 39 is an isometric view of a catheter having a brush electrode according to the present invention forming a linear or drag lesion on a section of tissue.

FIG. 40 is a cross-sectional view of the distal tip of a catheter having a brush electrode with an angled tip according to the present invention forming linear or drag surface lesion on a section of tissue.

FIG. 41 is a cross-sectional view of the distal tip of a catheter having a brush electrode with a pointed tip forming a deep lesion on a section of tissue.

FIG. 42 is a cross-sectional view of the distal tip of a catheter having a brush electrode with a rounded tip forming a shallow lesion on a section of tissue.

FIGS. 43-45 depict a brush electrode according to the present invention forming different-sized lesions based in part upon the amount of splay of the brush electrode.

FIG. 46 is a cross-sectional view of the distal tip of a catheter having a brush electrode with an exposed portion surrounded by a mesh fabric.

FIG. 47 is an isometric view of the brush electrode of FIG. 46 depicting the mesh fabric reducing the splaying of the filaments.

FIG. 48 is a fragmentary, isometric view in partial cross section of the distal end of a catheter with a shielded-tip brush electrode.

FIG. 49 is a fragmentary, elevation view in partial cross section of the shielded-tip brush electrode catheter depicted in FIG. 48.

FIG. 50 is a fragmentary, elevation view in cross section of the distal end of a catheter with an alternative shielded-tip brush electrode.

FIG. 51 is a cross-sectional view taken along line 51-51 of FIG. 50.

FIG. 52 is a fragmentary, isometric view in partial cross section of a solenoid-type, coil-wire sheath, brush electrode catheter according to the present invention.

FIG. 53 depicts the brush electrode catheter of FIG. 52 in partial cross section with the distal end of the catheter deflected.

FIG. 54 is a fragmentary, elevation view in partial cross section of a brush electrode catheter with a braided-wire sheath according to the present invention.

FIG. 55 is a fragmentary, elevation view in partial cross section of a brush electrode catheter with a straight-wire sheath according to the present invention.

FIG. 56 is a fragmentary, isometric view of a bipolar brush electrode catheter according to another embodiment of the invention.

FIG. 57 is a fragmentary, isometric view of the bipolar brush electrode catheter of FIG. 56, wherein the distal end of the brush electrode is deflected.

FIG. 58 is a fragmentary, schematic view of a bipolar brush electrode catheter within a vein forming a lesion on a section of the veinal wall.

FIG. 59 is a fragmentary, schematic view of a bipolar brush electrode catheter forming a lesion on a section of tissue.

FIG. 60 is a fragmentary, schematic view of a bipolar brush electrode catheter with a balloon return electrode forming a lesion on a section of tissue.

### DETAILED DESCRIPTION OF THE INVENTION

Several embodiments of a catheter with a conforming electrode according to the present invention are depicted in the figures. As described further below, the conforming electrode of the present invention provides a number of advantages, including, for example, the formation of deep lesions in tissue while reducing the formation of undesirable charring of the surface tissue; the application of therapeutic RF energy for therapeutic effects at a reasonable and manageable level; the achievement of greater electrode-tissue contact; a reduction in the volume of conductive fluid (e.g., saline) introduced into the bloodstream; and the mitigation of electrode-tissue contact problems. The present invention facilitates the formation of a deep lesion or incision in a shorter period of time than required by other ablation devices. It also provides the ability to create lesions in highly perfused tissue or in fluid-rich environments. The brush electrode 10 facilitates enhanced tissue contact in difficult environments (e.g., during ablation of a contoured or trabecular tissue surface on a beating heart) by readily conforming to surface contours.

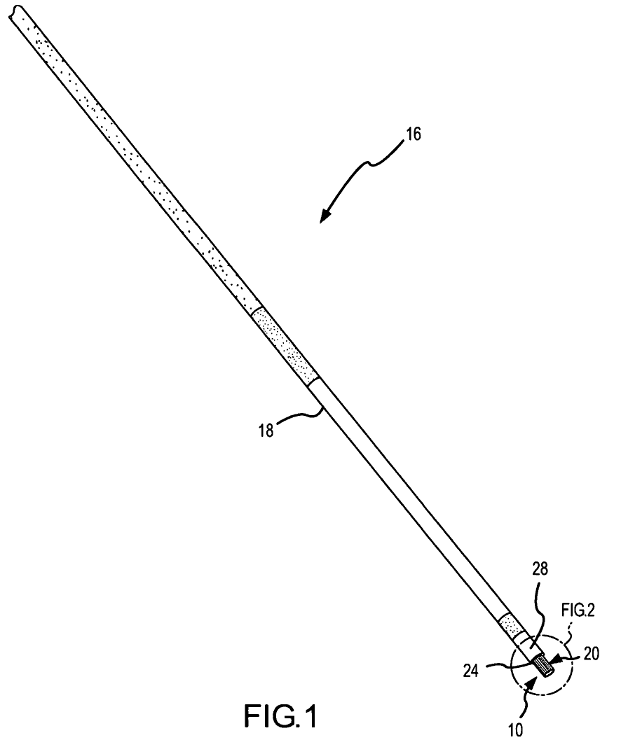

FIG. 1 is an isometric view of one embodiment of a catheter 16 having a brush electrode 10 according to the present invention. As depicted in this figure, the catheter comprises a catheter shaft with an outer sheath 18. In the embodiment depicted in FIG. 1, the outer sheath is formed from sections of different material (e.g., in the embodiment depicted FIG. 1, five different sections comprise the outer sheath). These sections of different material enable the catheter 16 to have, for example, different mechanical properties (e.g., flexibility) at different locations along the catheter shaft. The outer sheath 18 may or may not comprise these sections of different material depending upon the intended application for the catheter. Additionally, the distal end 24 of the outer sheath 18 may include a conductive or nonconductive base 28. Although the outer sheath 18 depicted in FIG. 1 has a circular cross section, the cross section of the outer sheath may be other than circular for all of the embodiments described herein.

As shown in FIGS. 1-5, the brush electrode 10 is provided at a distal end 24 of the outer sheath 18. (As used herein, “proximal” refers to a direction away from the body of a patient and toward the clinician. In contrast, “distal” as used herein refers to a direction toward the body of a patient and away from the clinician.) The brush electrode 10 is composed of a plurality of filaments 26 arranged longitudinally in a bundle. As shown particularly in FIG. 5, the bundle of filaments 26 is partially inserted into a lumen 44 defined by the outer sheath 18 at the distal end of the outer sheath 18. That portion of the filaments 26 within the lumen 44 may be referred to as an embedded portion 22, and that portion of the filaments 26 extending from the distal end 24 of the outer sheath 18 may be referred to as an exposed portion 20. The exposed portion 20 of the brush electrode 10 may project a few millimeters from the distal end 24 of the outer sheath 18. The distance that the exposed portion 20 of the brush electrode 10 extends from the distal end 24 of the outer sheath 18 varies depending upon a number of factors including the composition of the filaments 26 comprising the brush electrode 10 and the particular area to be treated with the brush electrode 10. As explained further below, the flexible brush electrode 10 provides enhanced tissue contact, particularly for use on contoured or trabecular surfaces.

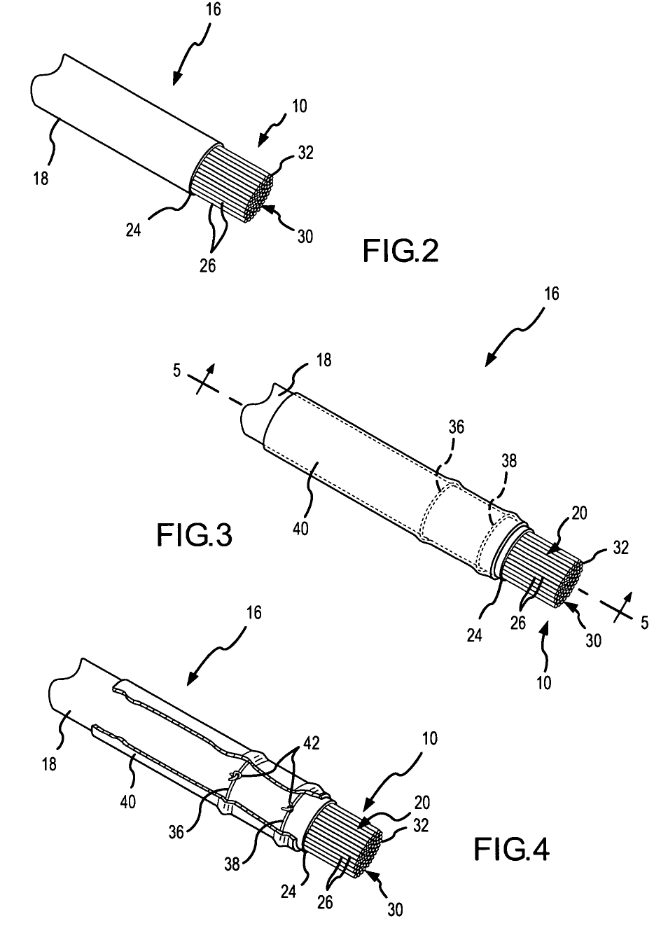

FIG. 2 is an enlarged view of the circled region of FIG. 1. As clearly shown in FIG. 2, the brush electrode 10 according to this embodiment has a relatively flat working surface 30 at the distal tip 32 of the brush electrode 10. In other words, in this depicted embodiment, each of the filaments 26 in the exposed portion 20 extend approximately the same distance from the distal end 24 of the outer sheath 18. Thus, the distal tip 32 of the brush electrode 10 provides a relatively flat working surface 30 composed of the longitudinal ends of the filaments 26. The outer sheath 18 of the catheter 16 provides mechanical support for the filaments 26 and may also provide electrical shielding.

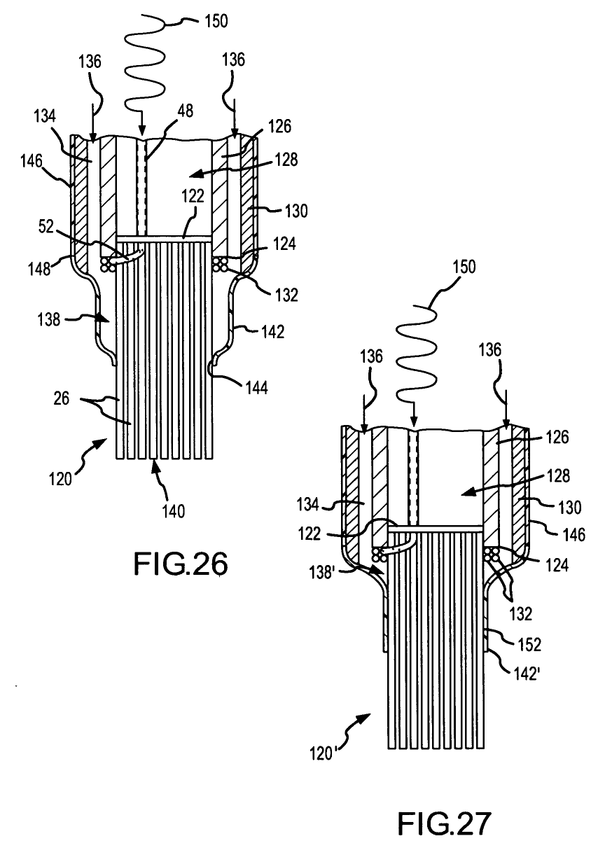

The brush electrode 10 may be composed of a bundle of bristles or filaments 26 that each may be constructed from a variety of different materials. Such materials may include nonconductive materials, semi-conductive materials, and conductive materials. For example, the filaments 26 may be formed from metal fibers, metal plated fibers, carbon compound fibers, and other natural materials. Very thin carbon fibers may be used, or relatively thicker, but less conductive, Thunderon® acrylic fibers (Nihon Sanmo Dyeing Company Ltd. of Kyoto, Japan) may be used for the brush electrode filaments 26. Nylon fibers coated with conductive material may also be used. Filaments 26 constructed from metal plated fibers, for example, coated nylon fibers, may have flattened areas around their outer surfaces, resulting in the filaments 26 having noncircular cross-sectional shapes. The filaments 26 may be insulated from each other, or they may be in electrical contact with each other. As explained further below, conductive or nonconductive fluids 34 may flow interstitially within the filaments 26 themselves (see, e.g., FIG. 5) or along the outer surface of the filaments (see, e.g., FIG. 26).

Once the distance that the filaments 26 extend from the distal end 24 of the other sheath 18 is set to a desired length, the bundle of filaments 26 comprising the brush electrode 10 may be fixed to the outer sheath 18. FIGS. 3-5 depict one technique for fixing or anchoring the brush electrode 10 relative to the outer sheath 18 using sutures 36, 38. In FIG. 3, a proximal suture 36 and a distal suture 38 are shown in phantom under a section of shrink tube 40 surrounding the outer surface of the outer sheath 18. The shrink tube 40 protects the sutures 36, 38 mitigating possible snags that may occur due to the presence of the sutures 36, 38, and makes it easier to manipulate the catheter 16. FIG. 4 is similar to FIG. 3, but depicts a portion of the shrink tube 40 broken away to reveal a portion of the two sutures 36, 38. The suture knots 42 are clearly visible in FIG. 4.

FIG. 5 is an isometric, cross-sectional view of the outer sheath 18 depicted in FIGS. 3 and 4, taken along line 5-5 of FIG. 3. The proximal suture 36 may be used to set the insertion depth of the embedded portion 22 of the brush electrode 10 in the distal end 24 of the outer sheath 18. In this figure, the distal suture 38 pierces the embedded portion 22 of the filaments 26 and thereby restricts movement of the brush electrode 10 relative to the outer sheath 18 of the catheter 16. In the embodiment depicted in FIG. 5, conductive fluid 34 is shown flowing through the lumen 44 of the outer sheath 18 from a fluid source, e.g., a pump and reservoir in a base unit (not shown), to the brush electrode 10. In an alternative embodiment, the lumen 44 depicted in FIG. 5 may comprise a plurality of separate lumen. When the conductive fluid 34 flows through the brush electrode 10, it creates a wet-brush electrode in which impinging jets of fluid traveling interstitially impact the tissue 46 (see, e.g., FIGS. 40-42) at the tissue-electrode interface, which makes it easier to control excessive temperature at the interface. Wet-brush electrodes are discussed further below.

FIG. 5 also clearly depicts a primary conductor 48 having an insulated portion 50 and an uninsulated portion 52. The primary conductor 48 carries RF energy from an energy source to the brush electrode 10. As depicted in FIG. 5, the primary conductor 48 extends within the fluid-carrying lumen 44 of the catheter 16, along a longitudinal axis 54 of the catheter 16. The primary conductor 48 may comprise, for example, insulated copper wire with an uninsulated portion 52 in electrical contact with the brush electrode 10. In this embodiment, the uninsulated portion 52 of the primary conductor 48 is looped or noosed around the filaments 26 comprising the brush electrode 10 at a connection point 56 (FIG. 7). At the loop or noose 58, RF energy is transferred from the primary conductor 48 to the conductive filaments 26 of the brush electrode 10. In this embodiment, the uninsulated portion 52 of the primary conductor 48 is connected with the embedded portion 22 of the brush electrode 10 so that the connection between the primary conductor 48 and the brush electrode 10 is protected within the outer sheath 18 of the catheter 16.

Also clearly visible in FIG. 5 is an embedded or secondary lead 60, which extends substantially parallel to the primary conductor 48. A distal end 62 of the secondary lead 60 becomes embedded with the filaments 26 of the brush electrode 10. As discussed further below in connection with, for example, FIG. 37, the secondary lead 60, when present, may be operatively connected to some type of sensor embedded in the brush electrode 10 (e.g., a thermal sensor 64, an ultrasound sensor 66, or a pressure sensor 68). The brush electrode 10 depicted in FIG. 5 acts as a surface-cooled electrode 10.

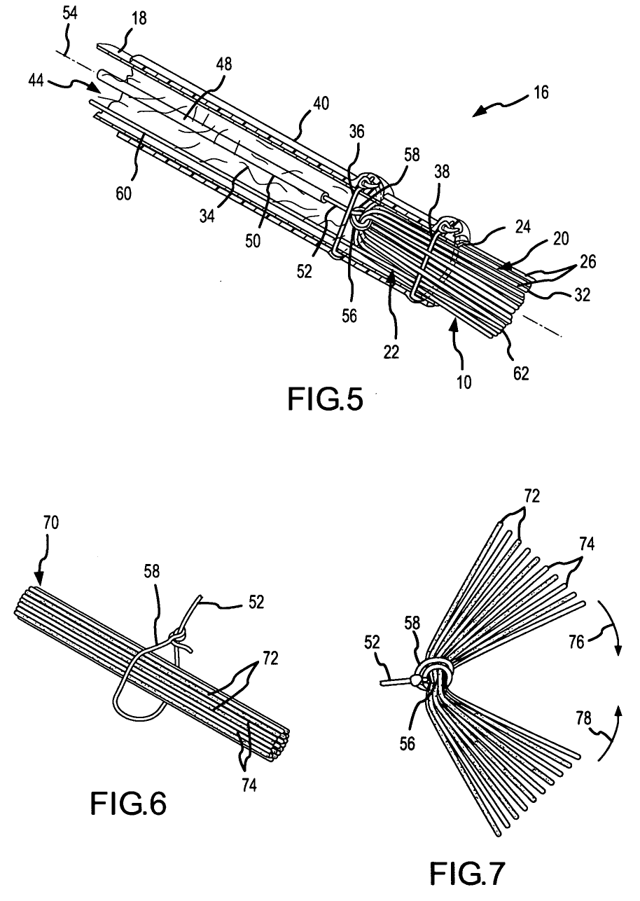

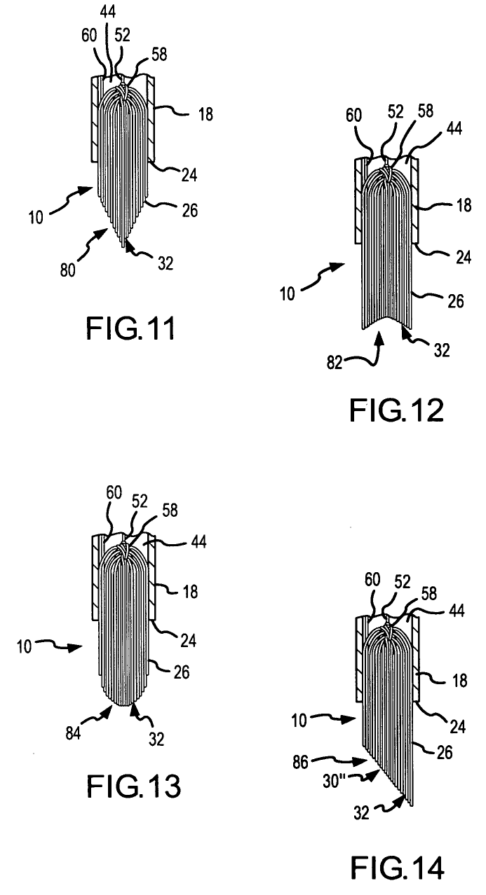

FIGS. 6 and 7 depict possible steps for forming the brush electrode 10 depicted in FIGS. 1-5. In FIG. 6, a bundle 70 of conductive filaments 72 and nonconductive filaments 74 is formed by using the uninsulated portion 52 of the primary conductor 48 to bind or tie together the filaments 70. In FIG. 6, the uninsulated portion 52 forms a noose around the bundle of filaments 70, but is not yet tightened or snugged against the bundle 70. In FIG. 7, the uninsulated portion 52 of the primary conductor 48 is snuggly noosed around the connection point 56 at approximately the mid-section of the bundle of filaments 70 that will ultimately form the brush electrode 10. The conductive filaments 72 and the nonconductive filaments 74 are then bent around the connection point 56 in the direction of the arrows 76, 78 depicted in FIG. 7. Once the filaments 70 are folded upon themselves about the connection point 56, they are inserted into the distal end 24 of the outer sheath 18 and positioned relative to the distal end 24 of the outer sheath 18 so that the desired amount of the filaments 70 extends from the distal end 24 of the outer sheath 18 and forms the exposed portion 20 of the brush electrode 10. The ends of the filaments 70 may then be trimmed, if desired, to create a desired shape for the working surface 30 at the distal end 32 of the brush electrode 10 (see, e.g., FIGS. 11-14).

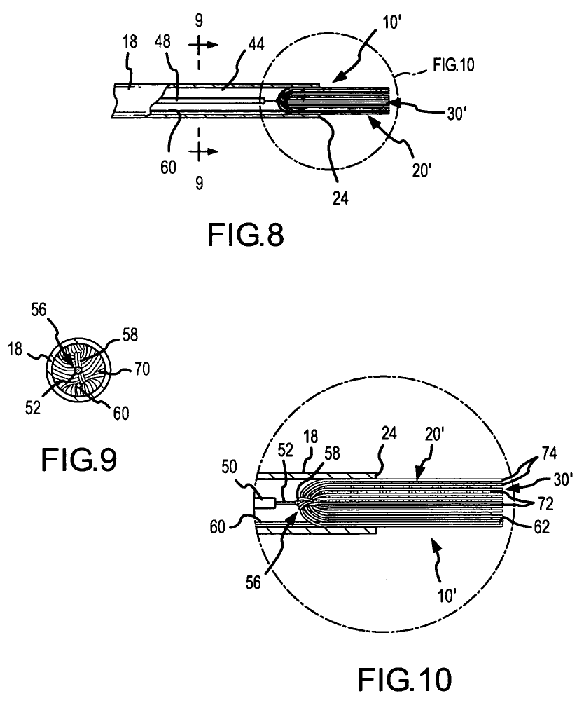

FIGS. 8, 9, and 10 depict an alternative embodiment of a brush electrode 10′. This standoff brush electrode 10′ includes an exposed portion 20′ with a working surface 30′, wherein the longitudinal ends of the conductive filaments 72 are not flush with the longitudinal ends of the nonconductive filaments 74. As shown to better advantage in FIG. 10, which is an enlarged view of the circled region of FIG. 8, in this alternative embodiment of the brush electrode 10′, the conductive filaments 72 are interspersed among relatively longer nonconductive filaments 74. The relatively longer nonconductive filaments 74 prevent the conductive filaments 72 from directly touching the tissue 46 when the working surface 30′ of the brush electrode 10′ is placed normal to the tissue 46 being treated. With this brush electrode 10′ configuration and substantially perpendicular orientation of the working surface 30′ relative to the tissue 46 being treated, the brush electrode 10′ acts as a virtual electrode. If the perpendicular orientation can be maintained, there is no direct contact between the conductive filaments 72 and the tissue 46, and the conductive fluid 34 (see FIG. 5) flowing through the lumen 44 of the outer sheath 18 makes the electrical contact at the brush-tissue interface. Although FIGS. 8 and 10 depict each of the conductive filaments 72 as being shorter than each of the nonconductive filaments 74, the electrical characteristics of the brush electrode 10′ may be adjusted by having some conductive filaments 72 extend to the working surface 30′ at the tip of the brush electrode 10′, if desired.

FIG. 9 is a cross-sectional view taken along line 9-9 of FIG. 8 and clearly depicts the bundled filaments 70 at the connection point 56 between the filaments 70 and the uninsulated portion 52 of the primary conductor 48. The secondary lead 60 is also visible in FIG. 9. In this embodiment, it is possible to adjust the fluid and electrical contact at the brush-tissue interface through appropriate selection of the conductive and nonconductive filaments 72, 74. Since this configuration of the brush electrode 10′ performs most effectively when placed normal or perpendicular to the tissue 46, a relatively short exposed portion 20′ for the brush electrode 10′ may be desirable with relatively stiff filaments (e.g., Thunderon® filaments).

FIGS. 11-14 depict alternative shapes for the filaments 26 comprising the distal tip 32 of the brush electrode 10. The various tip configurations may provide advantages for special applications of brush electrodes 10. FIG. 11 depicts a triangular distal tip 80 with filaments on opposing sides cut at corresponding angles to create a blade-like tip. In an alternative embodiment (not shown), the distal tip may be conical with its longest filaments proximal to the longitudinal axis 54 of the catheter 16 (see FIG. 5). These particular configurations may be advantageous for point applications of therapeutic energy, or for creating an incision in the tissue. In FIG. 12, the working surface of the electrode tip has a concave portion or channel 82. The concave-tip embodiment depicted in FIG. 12 is beneficial for wrap-around applications and provides advantages when treating curved surfaces like the surface of a blood vessel. FIG. 13 depicts an arched tip 84. The tip may be similarly a convex or domed tip. This particular configuration is beneficial, for example, when reaching into troughs or depressions on a trabecular or contoured surface. In an alternative embodiment (not shown), the distal tip 32 may be bowl-shaped, wherein the filaments 26 about the perimeter of the brush electrode 10 are longer than the filaments 26 proximal to the longitudinal axis 54 of the catheter 16. In FIG. 14, the brush electrode 10 has a wedge-shaped tip 86. The wedge-shaped tip 86 facilitates angular placement and increases the area of the working surface 30″. The brush electrodes 10 are depicted in many of the drawings with circular cross sections, but may have different cross-sectional configurations.

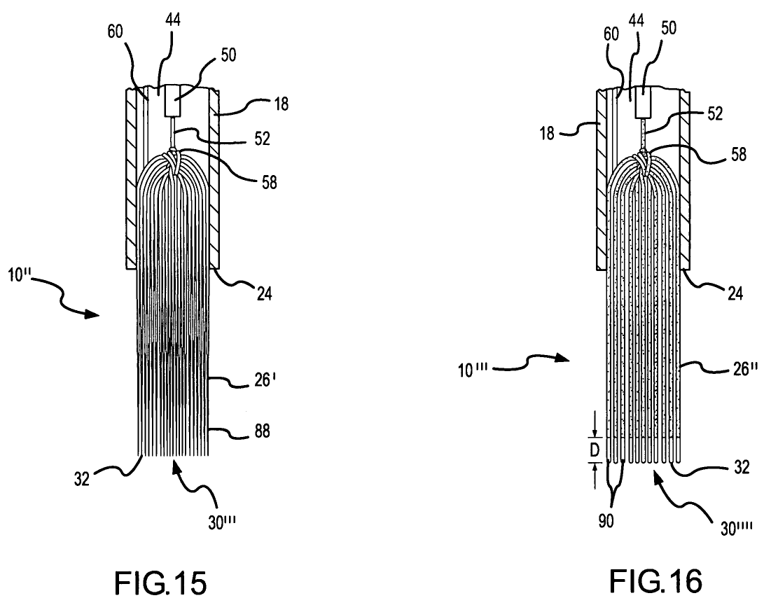

FIG. 15 depicts an example of a brush electrode 10″ having continuously varying conductivity along the longitudinal axes of the filaments 26′. In particular, the brush electrode 10″ comprises tapered filaments 26′. In this alternative embodiment, the individual filaments 26′ of the brush electrode 10″, or a portion thereof, have a tapered portion 88 gradually formed toward their distal ends at the distal tip 32 of the brush electrode 10″. In other words, at the distal end 24 of the outer sheath 18, the filaments 26′ have larger cross-sectional areas than they have at the distal tip 32, adjacent to the working surface 30′″ of the brush electrode 10″. The filaments 26′ are thus more conductive adjacent to the distal end 24 of the outer sheath 18 and less conductive along the tapered portion 88 due to the reduction in cross-sectional area. Since the filaments 26′ are more conductive adjacent to the distal end 24 of the outer sheath 18, current flow to the less conductive fluid wetting the brush electrode 10″ from the lumen 44 of the outer sheath 18 is minimized. When less of the RF energy flows into the conductive fluid 34 adjacent to the distal end 24 of the outer sheath 18, energy transfer into the conductive fluid 34 and the concomitant heating of the conductive fluid 34 before it contacts the surface of the tissue 46 is minimized. Along the tapered portion 88 of the filaments 26′ depicted in FIG. 15, the conductivity of the filaments 26′ may be matched to the conductivity of the fluid 34 to create a relatively uniform electric field at the brush-tissue interface.

Although not depicted, the tapered portion 88 shown in FIG. 15 could be an inverse taper (i.e., the cross-sectional area of the filaments increases from the distal end 24 of the outer sheath 18 to the distal tip 32 of the brush electrode), which may be advantageous for certain applications. It should be noted that, in order to vary the conductivity along the length of the filaments, the filaments may also be coated or plated with materials having different or varying electrical conductivity. For example, the filaments, whether tapering or not, could be coated with conductive material. The conductive material coating the filaments in the region most closely adjacent to the distal end 24 of the outer sheath 18 may be more conductive than the coating on the portion of the filaments most closely adjacent to the distal tip 32 of the filaments themselves. Thus, the conductivity of the filaments would be greater near the distal end 24 of the outer sheath 18 than near the distal tip 32 of the filaments, even though the cross-sectional areas of the filaments may not change substantially longitudinally along the filaments toward the distal tip 32. Although not specifically shown in the figures, the conductivity of all of the disclosed filaments may also vary radially rather than, or in addition to, varying longitudinally. In other words, the conductivity of the filaments may vary as one moves from the center of the filaments to the surface of the filaments.

FIG. 16 depicts a brush electrode 10′″ in which the conductivity of the filaments 26″ varies discontinuously. In particular, FIG. 16 depicts filaments 26″ that are conductive except at their distal ends at the distal tip 32 of the brush electrode 10′″. The distal end of each filament 26″ includes a nonconductive tip 90. These nonconductive tips 90 provide a stand-off distance D when the working surface 30″″ of the brush electrode 10′″ is placed substantially perpendicular to the tissue 46 being treated since the conductive portions of the filaments 26″ do not actually touch the tissue 46 in this embodiment. Similar to the embodiment depicted in FIGS. 8-10, the conductive fluid 34 passes through the lumen 44 of the catheter 16 and wet the filaments 26″ of the brush electrode 10′″. The conductive fluid 34 carries the RF energy over the stand-off distance D and to the tissue 46, and thereby acts as a virtual electrode. It should be noted that, although the embodiment depicted in FIG. 16 shows each of the conductive filaments 26″ having a nonconductive tip 90, in an alternative embodiment some of the conductive filaments 26″ may extend all the way to the working surface 30″″ of the brush electrode 10′″ and thus would, in fact, contact the tissue 46 during use of the brush electrode 10′″.

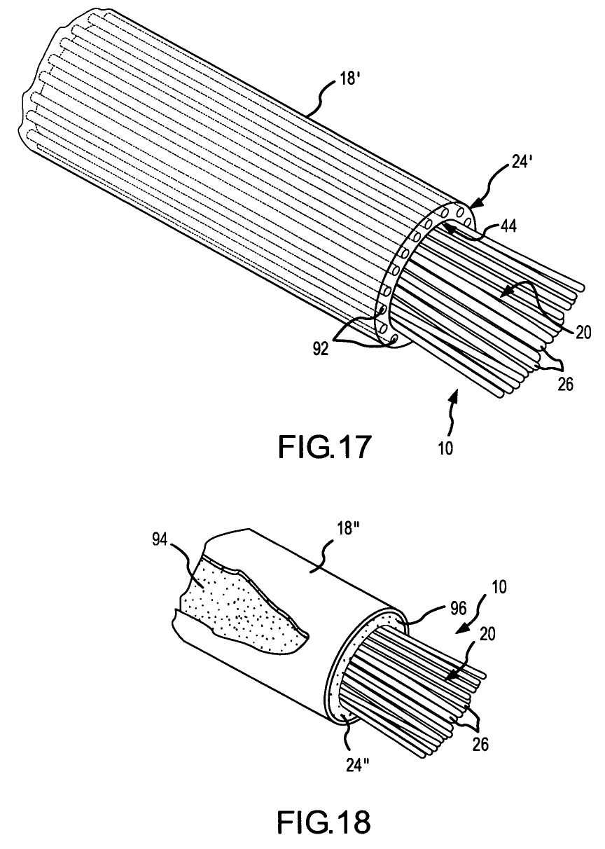

FIG. 17 depicts an embodiment of the outer sheath 18′ having a concentric ring of tubes 92 within the wall of the outer sheath 18′ that defines the central lumen 44 through which the brush filaments 26 extend. The circumferential ring of tubes 92 around the lumen 44 may be used to carry conductive or nonconductive fluid, including therapeutic fluid or medicine. The embedded tubes 92 depicted in this figure could define spiral or helical paths toward the distal end 24′ of the outer sheath 18′, similar to the paths or channels 104 described below in connection with FIGS. 19 and 20.

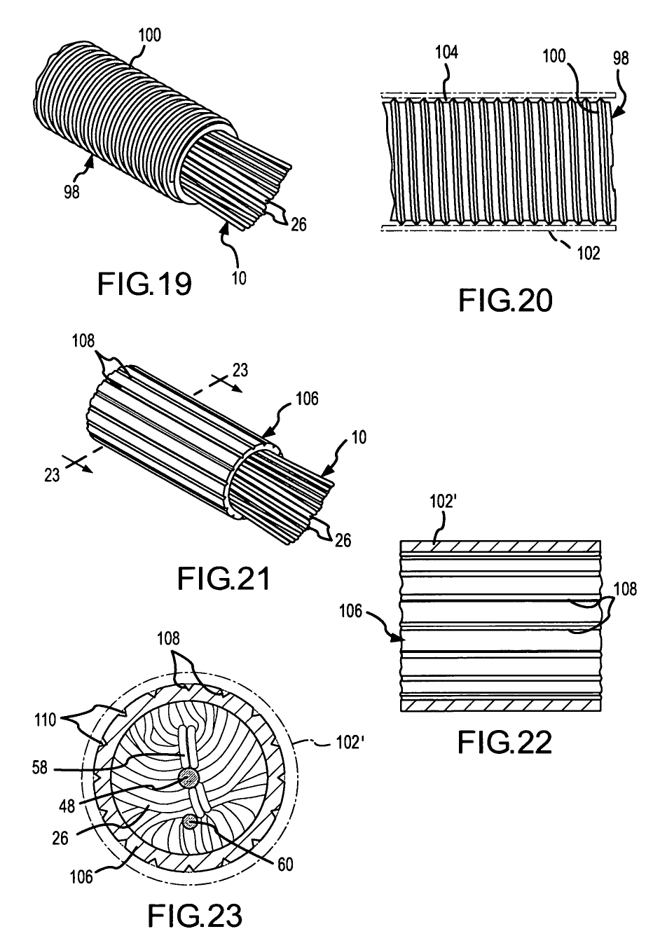

FIG. 18 depicts an embodiment wherein a porous sheath 94 surrounds the filaments 26 of the brush electrode 10 adjacent to the exposed portion 20 of the brush electrode 10. An outer covering 18″, possibly a thin, unitary extension of the outer sheath, may be placed around the outer cylindrical surface of the porous sheath 94. An angular ring of material 96 may be exposed at the distal end 24″ of the porous sheath 94 adjacent to the exposed portion 20 of the brush electrode 10.

FIG. 19 is a fragmentary, isometric view of an embodiment wherein a threaded sheath 98 surrounds the filaments 26 of the brush electrode 10. The threaded sheath 98 has a spiral or helical ridge 100 on its outer surface. As shown to good advantage in FIG. 20, when the threaded sheath 98 is inserted into an outer covering 102, possibly a thin, unitary extension of the outer sheath (shown in cross-section), a helical flow channel 104 is created between the threaded sheath 98 and the outer covering 102. Conductive fluid, nonconductive fluid, or medication may be delivered to the tissue adjacent to the brush electrode 10 via this flow channel 104.

FIG. 21 is a fragmentary, isometric view of another embodiment, wherein a grooved sheath 106 surrounds the filaments 26 of the brush electrode is. The grooved sheath 106 has a plurality of longitudinally-extending grooves or cuts 108 formed on its outer surface, adjacent to the exposed portion of the brush electrode 10. FIG. 22 is a fragmentary view of a section of the grooved sheath 106 depicted in FIG. 21, surrounded by an outer covering 102′, possibly a thin, unitary extension of the outer sheath (shown in cross-section) to create a plurality of longitudinally-extending flow channels 110 (shown to better advantage in FIG. 23) between the grooved sheath 106 and the covering 102. As shown in FIG. 23, when the grooved sheath 106 is inserted into the outer covering 102′ (shown in phantom and cross-section), the plurality of longitudinally-extending flow channels 110 are created between the grooved sheath 106 and the outer covering 102′. Again, conductive fluid, nonconductive fluid, or medication may be delivered to the tissue 46 adjacent to the brush electrode 10 via these flow channels 110.

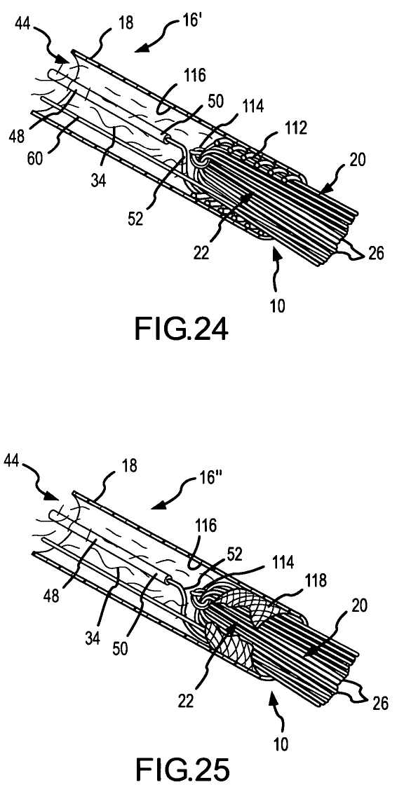

FIGS. 24 and 25 depict alternative mechanical interfaces between the filaments 26 of the brush electrode 10 and the primary conductor 48. FIG. 24 is similar to FIG. 5, but depicts an isometric, cross-sectional view of a catheter 16′ wherein the exposed portion 52 of the primary conductor 48 makes electrical contact with the brush filaments 26 via an energy transfer coil or spring 112 surrounding at least the embedded portion 22 of the brush electrode 10. In this embodiment, the RF energy is transferred to the brush electrode 10 over a large surface area (i.e., over the entire inner surface area of the coil 112). Thus, less damage to the filaments 26 may occur in this embodiment than may occur in the embodiment depicted in FIG. 5, wherein all of the RF energy is transferred from the uninsulated portion 52 of the primary conductor 48 to the brush electrode 10 at the single connection point 56. As depicted in FIG. 24, a loop of wire 114 may be present to help collect and stabilize the filaments 26 during assembly of the catheter 16′. This loop of wire 114 may be anchored to, for example, the inner surface 116 of the outer sheath 18. As previously described, a secondary lead 60 may also be present in the lumen 44 of the outer sheath 18.

FIG. 25 is similar to FIGS. 5 and 24, but depicts an isometric, cross-sectional view of a catheter 16″ wherein the primary conductor 48 makes electrical contact with the filaments 26 of the brush electrode 10 via an energy transfer mesh or fabric 118 surrounding at least the embedded portion 22 of the brush electrode 10. This embodiment has the same advantages previously described for the embodiment depicted in FIG. 24.

FIG. 26 is a cross-sectional view of a first embodiment of a shielded-tip brush electrode 120. In this embodiment, the uninsulated portion 52 of the primary conductor 48 is looped around the outer surface of the brush electrode after passing through a mechanical interface 122 supporting the filaments 26 of the shielded-tip brush electrode 120 adjacent to the distal end 124 of an inner sheath 126. Since fluid may or may not travel through the lumen 128 of the inner sheath 126, the mechanical interface 122 may or may not be porous. It should be noted that, although the filaments 26 are shown as extending only into the distal end 124 of the inner sheath 126, the filaments 26 may extend further into the inner sheath 126 and may even extend all the way to the proximal end (not shown) of the catheter.

In the embodiment depicted in FIG. 26, an outer sheath 130 surrounds the inner sheath 126. The inner sheath 126 houses the primary conductor 48 and supports the mechanical interface 122 for the filaments 26 of the brush electrode 120. The primary conductor 48 again includes an uninsulated portion 52 that transfers RF energy 150 to the conductive filaments 26 in the shielded-tip brush electrode 120. As mentioned, in this embodiment the uninsulated portion 52 of the primary conductor 48 forms loops or coils 132 around the circumference of the brush electrode 120. These loops or coils 132 increase the surface area through which the RF energy 150 is transferred, thereby providing more effective, and potentially less destructive, energy transfer to the brush electrode 120.

As shown in FIG. 26, the outer sheath 130 is placed around the inner sheath 126, but is radially and longitudinally offset from the inner sheath 126. The radial offset creates an annular gap or channel 134 between the inner sheath 126 and the outer sheath 130 through which conductive fluid may, for example, be introduced to the sides of the filaments 26. The conductive fluid, if present, would flow through the annular channel 134 in the direction of the arrows 136 shown at the top of FIG. 26. The longitudinal offset between the inner sheath 126 and the outer sheath 130 ensures that the channel 134 for the conductive fluid extends past the distal end 124 of the inner sheath 126 to the sides of the filaments 26. In this embodiment, the conductive fluid 34 would flow through the annular channel 134 between the inner sheath 126 and the outer sheath 130, past the coils 132 of uninsulated conductive wire 52, into an annular fluid jacket 138 surrounding a region of the brush electrode 120 adjacent to the distal ends of the inner sheath 126 and outer sheath 130, and then into the sides of the brush electrode 120 itself and through the interstitial gaps between the filaments 26 comprising the brush electrode 120. The RF energy 150 is thus carried by the conductive fluid 34 into the core of the brush electrode 120 and toward its working surface 140.

In this embodiment, a flexible polymer nipple or boot 142, defining an outer wall of the annular fluid jacket 138, also supports the filaments 26 in a ring 144 of direct contact extending around the perimeter of the bundle of filament 26. The flexible boot or nipple 142 may be porous. The flexible boot 142 may extend proximally about the outer surface of the outer sheath 130 and abut a corresponding distal edge 148 of the outer sheath 130 to form a smooth outer wall 146 around the edge 148. An annular layer of porous material or mesh fabric (not shown) may be placed in the annular fluid jacket 138 to keep the filaments 26 wetted and to help prevent splaying (see FIGS. 43-45) of the brush electrode 120.

FIG. 27 is similar to FIG. 26, but depicts a second embodiment of a shielded tip brush electrode 120′. The only differences between the embodiment depicted in FIG. 26 and the embodiment depicted in FIG. 27 are the size of the fluid jacket and the configuration of the flexible polymer nipple or boot that supports the brush filaments. In the embodiment depicted in FIG. 27, an alternative flexible polymer nipple or boot 142′ defines a smaller fluid jacket 138′ and supports the filaments 26 in a band of direct contact 152 extending around the perimeter of the bundle of filaments 26. The band of direct contact 152 supports the filaments 26 over a larger section of the outer surface of the brush electrode 120′ than does the ring of direct contact 144 depicted in FIG. 26. By adjusting the configuration of the flexible polymer nipple or boot 142′ in this manner, the amount of conductive fluid flowing into the brush electrode and the overall flexibility of the brush electrode can be manipulated.

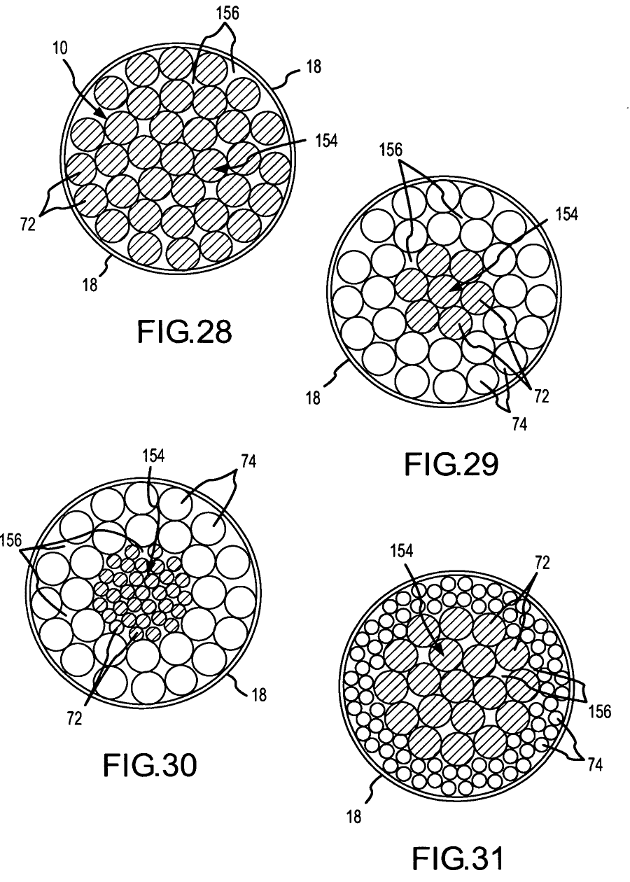

FIGS. 28-35 schematically depict different cross-sectional configurations for brush electrodes 10 according to the present invention. Interstitial spaces 156 are clearly visible in each of these figures. In FIGS. 28-31, the brush electrode 10 has a conductive core 154. In these four figures, the conductive filaments 72 are shown with cross hatching, and the nonconductive filaments 74 are shown without cross hatching. Thus, the brush electrode 10 depicted in FIG. 28 is fully conductive and does not have any nonconductive filaments 74. In each of the embodiments depicted in FIGS. 29-31, a conductive core 154 is shielded by a barrier of nonconductive filaments 74. In particular, FIG. 29 depicts a core of relatively large conductive filaments 72 surrounded by two rings of nonconductive filaments 74 of approximately the same size. In FIG. 30, a core 154 of relatively small conductive filaments 72 is surrounded by two rings of relatively large nonconductive filaments 74. In FIG. 31, a conductive core 154 of relatively large conductive filaments 72 is surrounded by two rings of relatively small nonconductive filaments 74.

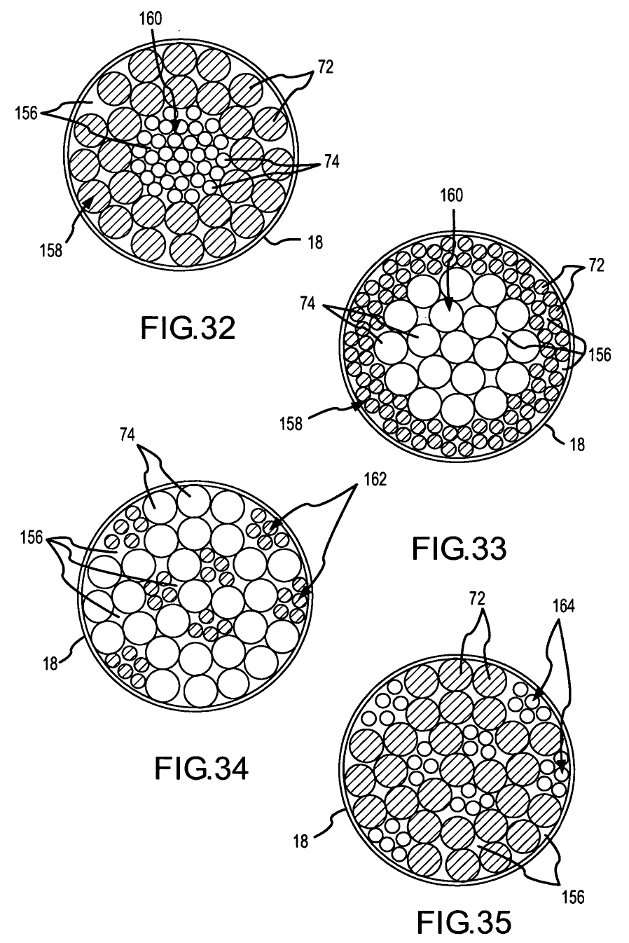

FIGS. 32 and 33 depict cross-sectional configurations for brush electrodes 10 that have conductive perimeters 158. Thus, in the embodiments depicted in FIGS. 32 and 33, a nonconductive core 160 of nonconductive filaments 74 is surrounded by conductive filaments 72. FIG. 32 depicts a core of relatively small nonconductive filaments 74 surrounded by a ring of relatively large conductive filaments 72. In FIG. 33, a core 160 of relatively large nonconductive filaments 74 is surrounded by a ring of relatively small conductive filaments 74.

In FIG. 34, conductive clusters 162 of relatively small filaments are interspersed among relatively large nonconductive filaments 74. The interspersed conductive clusters 162 may be interspersed in a specific pattern, pseudo randomly, or randomly among the nonconductive filaments 74 in order to achieve a desired electric field from the resulting brush electrode 10. In FIG. 35, nonconductive clusters 164 of relatively small filaments are interspersed among relatively large conductive filaments 72.

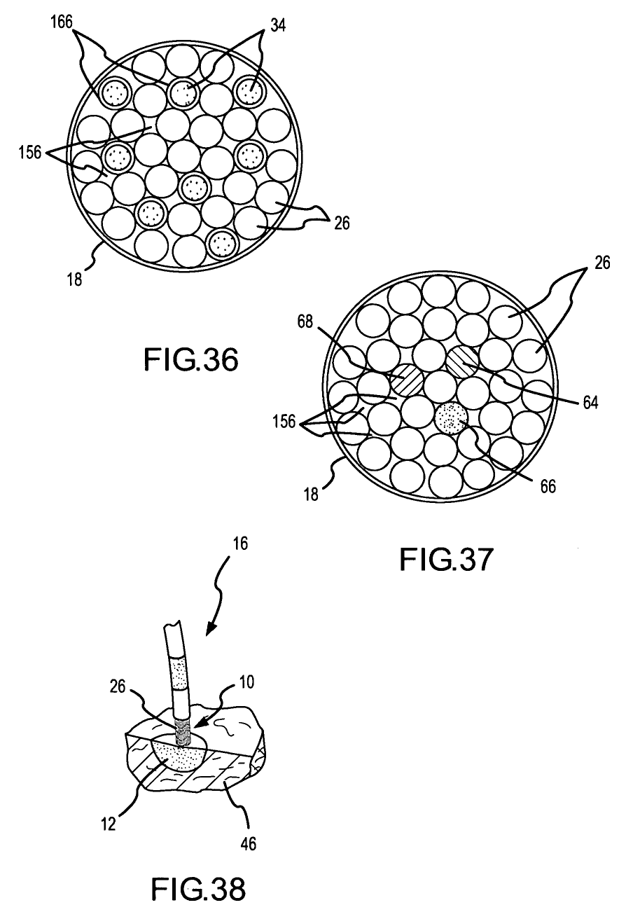

FIG. 36 is a cross-sectional view of a brush electrode 10 wherein some of the filaments housed within the outer sheath 18 are hollow or porous 166. Such hollow or porous filaments 166 may be used as conduits for conductive fluid 34, they may be used to supply therapeutic medications, and they may provide suction ports at the brush-tissue interface to control field smearing on the tissue surface. If the filaments are porous, they may retain a small amount of fluid in pores that are oriented at various angles to the longitudinal axis of the filaments. During an ablation procedure, some of the RF energy may dehydrate the porous filaments 166 before affecting the surrounding blood, particularly when the conductivity of the tissue lessens as the ablation procedure progresses. Thus, if excess RF energy is present during a procedure, that energy may harmlessly dehydrate the porous filaments 166 rather than negatively affecting the tissue being treated by the brush electrode or the blood in the area of that tissue. In the embodiment depicted in FIG. 36, the other filaments 26 may be conductive or nonconductive filaments.

FIG. 37 is a cross-sectional view of a brush electrode 10 having devices 64, 66, 68 embedded among the conductive and nonconductive filaments 26 housed in the outer sheath 18. The devices may include, for example, pressure sensors 68 to measure contact pressure between the brush electrode 10 and the tissue, thermal sensors 64 (e.g., a thermocouple) at the tip of the brush electrode 10 to sense the brush-tissue interface temperature, or fiber optic or ultrasound sensors 66 for in situ lesion identification and characterization. The devices may be operatively connected to equipment (not shown) at the proximal end of the catheter by secondary leads like the secondary lead 60 depicted in, for example, FIGS. 5 and 8-16.

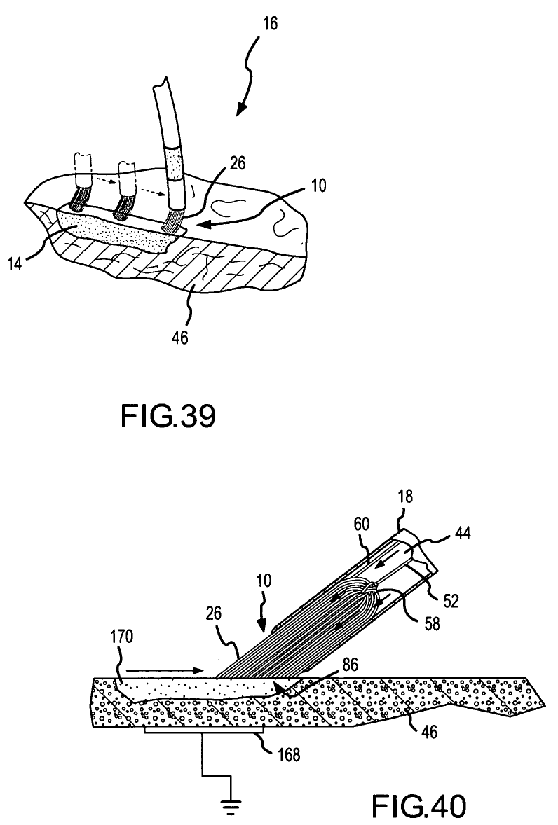

FIG. 38 is a fragmentary, isometric view of a catheter 16 having a brush electrode 10 according to the present invention forming a spot or point lesion 12 on a section of tissue 46. As shown in this figure, the brush electrode is placed against the tissue with its filaments 26 in contact with or in close proximity to the tissue 46. The conductive filaments 26 are connected to, for example, an RF source (not shown) and serve as the active electrode. When present, conductive fluid from a fluid source (not shown) flows through the lumen 44 (e.g., FIG. 5) of the catheter 16 and through the brush filaments 26 to the working surface at the brush tip, thereby creating a wet-brush electrode. The brush electrode 10 may also be dragged along the surface of a section of tissue 46 to create a continuous linear or drag lesion 14 as shown in FIG. 39.

FIG. 40 is a fragmentary, partial cut-away view of a catheter 16 having a brush electrode 10 with a wedge-shaped tip 86, e.g., according to FIG. 14 of the present invention, forming a surface lesion 170 on a section of tissue 46. As shown in this figure, the brush electrode 10 is placed against the tissue 46 with its filaments 26 in contact with or in close proximity to the tissue 46. The conductive filaments 26 are connected to an RF source (not shown) and serve as the active electrode. A return electrode 168 is affixed to another part of the patient's body with a large surface area, for example, the thigh, for dispersion of the current density and acts as the passive electrode to ground. When present, conductive fluid 34 (e.g., as shown in FIG. 5) from a fluid source (not shown) flows through the lumen 44 of the catheter 16 and through the brush filaments 26 to the working surface at the brush tip, thereby creating a wet-brush electrode. The brush electrode 10 can be localized on the tissue 46 to create a spot or point lesion, as shown in FIG. 38, or the brush electrode 10 may be dragged along the surface of the tissue 46 to create a continuous linear lesion, as shown in FIG. 39.

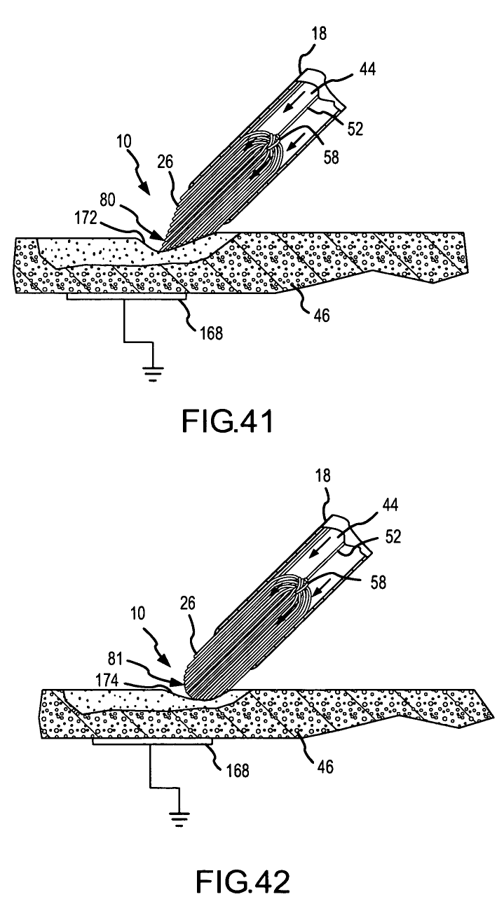

FIG. 41 is a fragmentary, partial cut-away view of a catheter 16 having a brush electrode 10 with a triangular tip 80, e.g., according to FIG. 11 of the present invention, forming a lesion 172 on a section of tissue 46. The embodiment of FIG. 41 is similar to that of FIG. 40 except for the resulting lesion created. In FIG. 41, a deep lesion 172 is formed as opposed to the surface lesion 170 of FIG. 40. The triangular tip 80 may be operated similar to a blade. If higher power electrosurgical energy were applied, the brush electrode 10 with the triangular tip 80 may function similar to an electrosurgical scalpel and create an incision in the tissue 46.

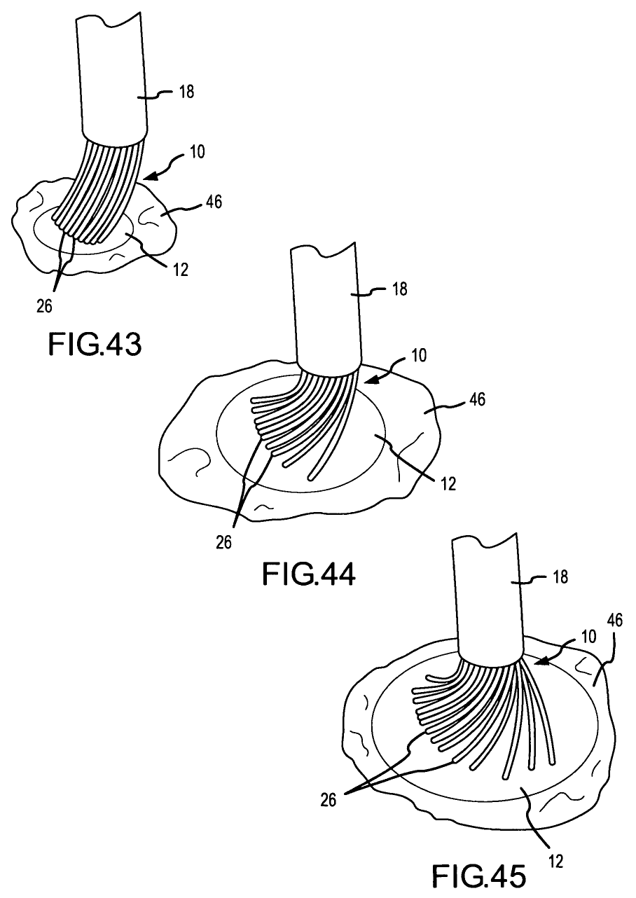

FIG. 42 is a fragmentary, partial cut-away view of a catheter 16 having a brush electrode 10 with a convex tip 84, e.g., according to FIG. 13 of the present invention, forming a lesion 174 on a section of tissue 46. The embodiment of FIG. 42 is similar to that of FIG. 40 except for the resulting lesion created. In FIG. 42, a shallow lesion 174 is formed as opposed to the surface lesion 170 of FIG. 40 or the deep lesion 172 of FIG. 41. The convex tip 84 provides more concentrated energy toward the center of the brush electrode 10. If higher power electrosurgical energy were applied, the brush electrode 10 with the convex tip 80 may function similar to a cauterizing or coagulation device to arrest bleeding, or alternately may be used to remove undesirable surface tissue 46, for example, a mole or tumor if used in an external application. Further, the convex tip makes better contact with uneven, undulating, or trabecular tissue surfaces to aid in the creation of uniform, linear lesions on such surfaces.

FIGS. 43-45 depict a brush electrode 10 according to the present invention forming different size spot lesions 12 based in part upon the amount of splay of the brush electrode 10. In FIG. 43, relatively light contact pressure is being used to press the brush electrode 10 against the tissue 46 while forming a lesion 12. This application of light pressure results in minimal splaying of the filaments 26 comprising the brush electrode 10, and thus a relatively small lesion 12 is formed. In FIG. 44, more pressure is being used to press the brush electrode 10 into contact with the tissue 46, resulting in relatively more splaying of the brush electrode 10. As long as the efficiency of the brush electrode 10 is not degraded too greatly by the splaying, a relatively larger lesion 12 may thus be formed by applying additional pressure to press the brush electrode 10 toward the tissue 46. In FIG. 45, even more contact pressure is being applied to the brush electrode 10 than is being applied in FIGS. 43 and 44, resulting in even more splaying of the brush electrode 10 and the formation of a relatively larger lesion 12 on the tissue 46 than is being formed in FIGS. 43 and 44.

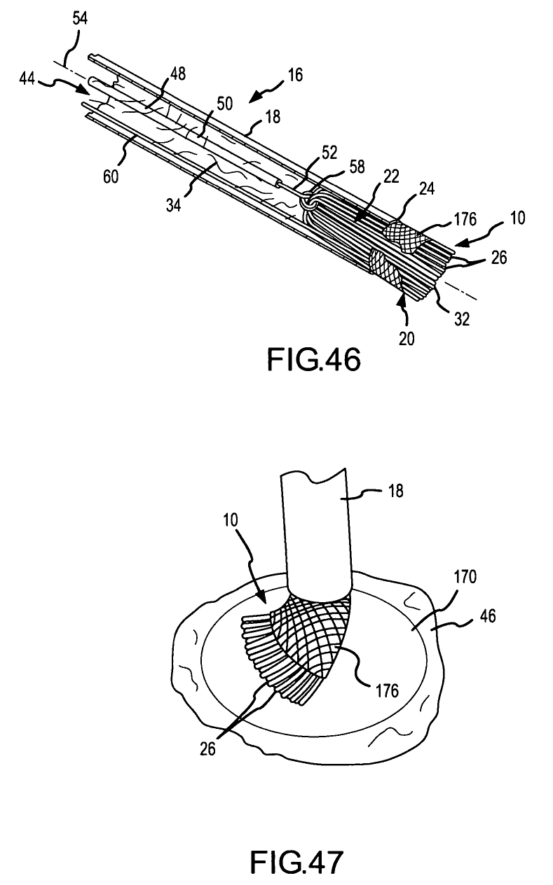

FIGS. 46 and 47 depict an alternative embodiment of a catheter 16 with a brush electrode 10, which includes a fabric or mesh jacket 176 about a proximal section of the exposed portion 20 of the filaments 26. The fabric or mesh jacket 176 acts to reduce the splaying of the filaments 26 if the application so requires. The fabric or mesh jacket 176 may be made of a conductive or nonconductive material depending upon the desired electric field effects for the brush electrode 10. The fabric or mesh jacket 176 may similarly be absorptive if it is desirable to retain fluid about the filaments 26 for cooling or other purposes. The proximal end of the fabric or mesh jacket 176 may be affixed to the distal end of the outer sheath 18 or it may extend into the lumen 44 of the outer sheath 18 and cover a section of the embedded portion 22 of the filaments 26. The fabric or mesh jacket 176 may also extend distally to the distal tip 32 of the brush electrode 10 completely restrict any splay in the filaments 26.

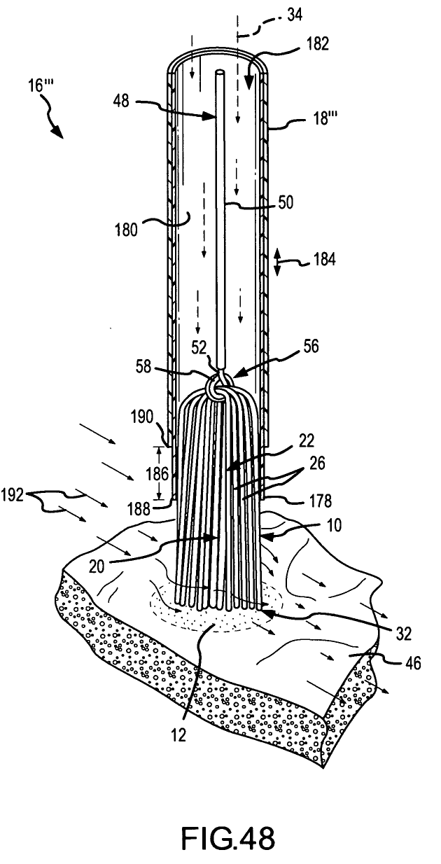

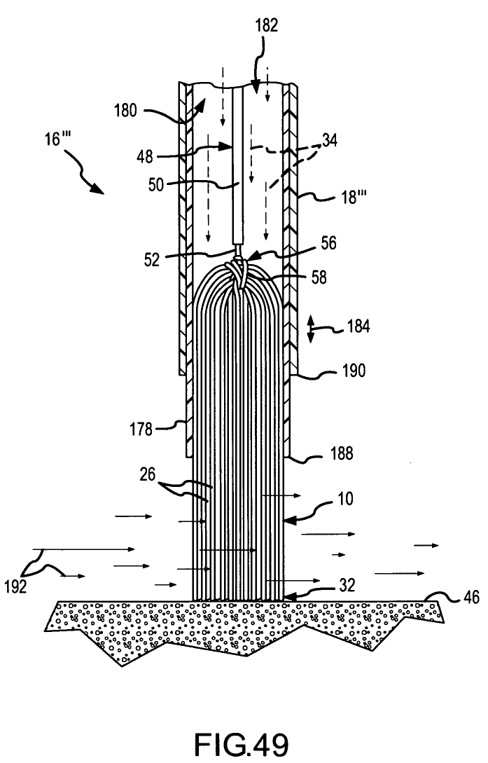

FIGS. 48 and 49 depict a another embodiment of a shielded-tip, brush electrode catheter 16′″ according to the present invention. FIG. 48 is a fragmentary, isometric view in partial cross section of the distal end the shielded-tip brush electrode catheter 16′″. Similarly, FIG. 49 is a fragmentary view in partial cross section of the same shielded—tip brush electrode catheter. As depicted in FIG. 48, the catheter 16′″ is forming a spot lesion 12 on a section of tissue 46. Ambient fluid 192 (e.g., blood) is shown schematically flowing around and among the filaments 26 of the exposed portion 20 of the brush electrode 10. In this embodiment, the brush electrode 10 is positioned longitudinally relative to an inner sheath 178, and may be fixed to the inner sheath 178 by, for example, sutures 36, 38 as shown in FIGS. 4 and 5. In FIG. 48, the brush electrode 10 frictionally engages the inside surface 180 of the inner sheath 178, but has not been directly attached to the inside surface 180 of the inner sheath 178.

As described in various other embodiments above, a conductor 48, which comprises an insulated portion 50 and an uninsulated portion 52, carries ablative energy to the connection point 56, where the uninsulated portion 52 of the conductor 48 transfers ablative energy to the brush filaments 26. In the embodiment depicted in FIG. 48, the uninsulated portion 52 of the conductor 48 is connected to the brush filaments 26 by loops 58 around the midsection of the filaments 26. Alternatively, any of the techniques above, among others, could be used to transfer ablative energy from the conductor 48 to the brush filaments 26. In FIGS. 48 and 49, conductive fluid 34 is flowing through the inner lumen 182 from the proximal end of the catheter 16′″ to the distal end 32 of the brush electrode 10.

As also shown in FIGS. 48 and 49, an outer sheath 18′″ surrounds at least a distal portion of the inner sheath 178. The outer sheath 18′″ may be longitudinally adjustable relative to the inner sheath 178 in the direction of arrow 184. In other words, the separation distance 186 between a distal end 188 of the inner sheath 178 and a distal end 190 of the outer sheath 18′″ may be adjustable by sliding the outer sheath 18′″ and the inner sheath 178 relative to each other. Alternately, the separation distance 186 may be fixed. By positioning the brush electrode 10 so that a desired length of brush filaments 26 comprises the exposed portion 20 of the brush electrode, and then adjusting the separation distance 186 between the distal end 188 of the inner sheath 178 and the distal end 190 of the outer sheath 18′″, the electric field at the brush/tissue interface may be adjusted.

If the exposed portion of the inner sheath 178 (i.e., the portion of the inner sheath 178 extending distally of the distal end 190 of the outer sheath 18′″) extends close to the distal end 32 of the brush electrode 10, the interaction between the ambient fluid 182 and the brush filaments 26 is greatly reduced. The exposed portion of the inner sheath 178 thus acts as a protective shield. This is similar to the effect achieved with the brush embodiments depicted in, for example, FIGS. 29-31. In each of FIGS. 29-31, a conductive core 154 is surrounded by rings of nonconductive filaments 74 to help shield the conductive filaments 72 comprising the conductive core 154. The inner sheath 178 or protective shield depicted in the embodiment of FIGS. 48 and 49 provides even greater protection for the conductive filaments 26 comprising the brush electrode 10. The nonconductive filaments 74 depicted in FIGS. 29-31 could be hydrophobic. Even if the nonconductive filaments 74 depicted in FIGS. 29-31 were hydrophobic, however, the solid shield depicted in FIGS. 48 and 49 would still provide a greater amount of shielding between the ambient fluid 192 and the conductive filaments 26 within the conductive core of the brush electrode 10.

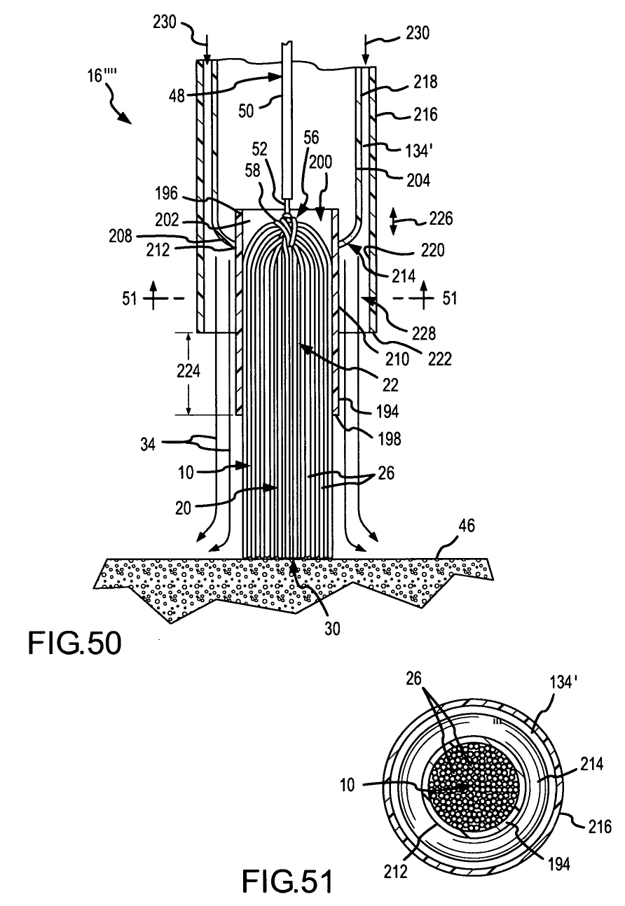

FIGS. 50 and 51 depict yet another embodiment of a shielded-tip brush electrode catheter 16″″ according to the present invention. In particular, FIG. 50 is a fragmentary, cross-sectional view of the distal end of this shielded-tip brush electrode catheter 16″″. Comparatively, FIG. 46 is a cross-sectional view taken along line 51-51 of FIG. 50. In this embodiment, a brush electrode 10 having an exposed portion 20 and an embedded portion 22 is frictionally mounted in an inner sheath 194. In FIG. 50, the inner sheath 194 is depicted as a stub sheath, which extends just proximally of the connection point 56 between the uninsulated portion 52 of the conductor 48 and the bundle of brush filaments 26. The inner sheath 194 may, however, extend to the proximal end (not shown) of the catheter 16″″. The inner sheath 194 depicted in FIG. 50 includes a proximal end 196 and a distal end 198, and the inner sheath 194 defines an inner lumen 200 in which the brush electrode 10 has been frictionally mounted. The brush electrode 10 may be (but need not be) affixed to the inside surface 202 of the inner sheath 194 (e.g., by sutures as depicted in FIGS. 4 and 5).

The brush electrode catheter 16″″depicted in FIG. 50 also includes an intermediate, supporting sheath 204. The intermediate, supporting sheath 204 defines an intermediate lumen 206 through which at least the insulated portion 50 of the conductor 48 is routed. The intermediate sheath 204 has an inwardly-curved, distal end 208 that is attached to an outside surface 210 of the inner sheath 194 along an annular attachment ring 212. The inwardly-curved, distal end 208 of the intermediate sheath 204 thus defines a blunt nose 214 that may be seen to good advantage in both FIGS. 50 and 51. The annular attachment ring 212 need not be a solid ring. If the ring 212 is other than solid, the intermediate lumen 206 will be in communication with the annular gap 134′ and/or the adjustable-length fluid jacket 228, both of which are discussed further below.

An outer sheath 216 it also depicted in FIGS. 50 and 51. The outer sheath 216 surrounds the intermediate, supporting sheath 204 and is radially offset from the intermediate, supporting sheath 204 to thereby define an annular gap or channel 134′ between an outside surface 218 of the intermediate, supporting sheath 204 and an inside surface 220 of the outer sheath 216. The outer sheath 216 has a distal end 222, which is depicted in FIG. 50 as being longitudinally offset from the distal end 198 of the inner sheath 194 by a separation distance 224. In this embodiment, the longitudinal position of the outer sheath 216 may be adjustable relative to the longitudinal position of the intermediate, supporting sheath 204 in the direction of arrow 226. In other words, it may be possible to adjust the separation distance 224 between the distal end 198 of the inner sheath 194 and the distal end 222 of the outer sheath 216 to ensure that the desired portion of the brush electrode 10 extends distally from the distal end 222 of the outer sheath 216.

An adjustable-length fluid jacket 228 is defined between the outside surface 210 of the inner sheath 194 and the inside surface 220 of the outer sheath 216. If conductive or nonconductive fluid 34 is introduced in the direction of arrows 230 into the annular gap or channel 134′, that fluid 34 would subsequently flow past the blunt nose 214 at the distal end of the intermediate, supporting sheath 204, and into the adjustable-length fluid jacket 228. This fluid 34 is then directed longitudinally over or along the exterior surface of the filament bundle 26 comprising the brush electrode 10 to form a cylindrical shield of cooling fluid around the outer circumference of the filament bundle 26. This flow of fluid 34 along the outer surface of the brush electrode 10 helps mitigate potential problems from direct contact between the ambient fluid 192 (see, e.g., FIGS. 48 and 49) and the brush filaments 26, particularly the conductive brush filaments. The ability to deliver cooling fluid 34 to the outer surface of the brush electrode 10 and to the tissue 46 being ablated also can enhance formation of a lesion 12 and mitigate collateral damage to the tissue 46 from potential overheating of the tissue surface during lesion formation.

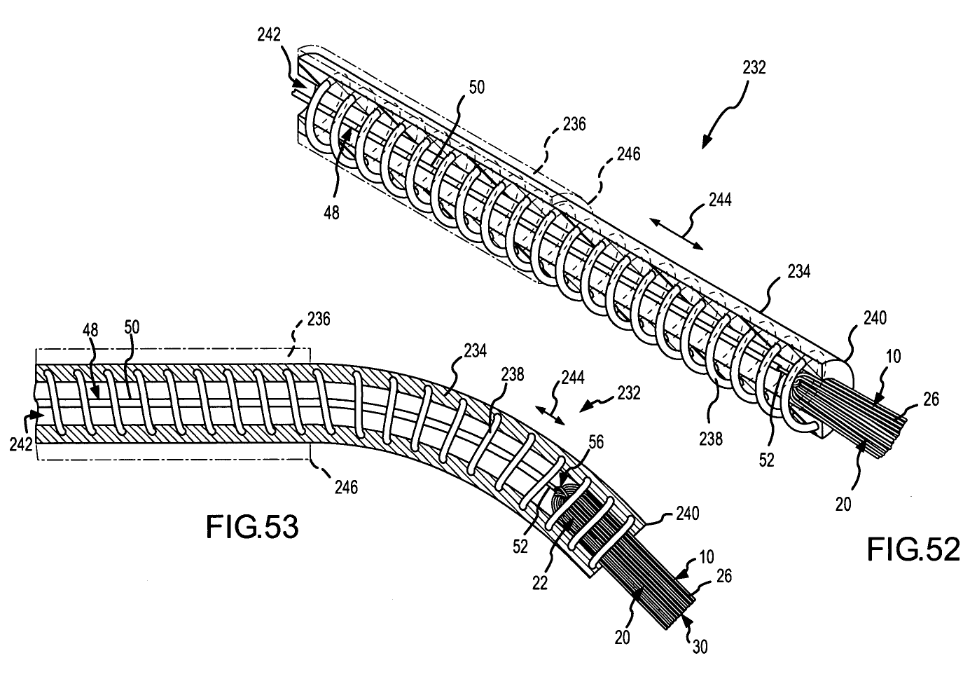

FIGS. 52 and 53 depict a solenoid-type, coil-wire sheath, brush electrode catheter 232. In particular, FIG. 52 is a fragmentary, isometric view in partial cross section of the solenoid-type, coil-wire sheath, brush electrode catheter 232. FIG. 48 depicts this brush electrode catheter 232 in partial cross section with the distal end of the catheter 232 deflected. As shown in these figures, this embodiment of the brush electrode catheter 222 comprises an inner sheath 234 and may include one or more outer sheaths (a single outer sheath 236 appears in phantom on FIGS. 47 and 48). As shown, a coil wire 238 is embedded in the material comprising the inner sheath 234. This embedded coil wire 238 may extend completely to a distal end 240 of the inner sheath 234 as shown in FIGS. 52 and 53, and it may extend any desired distance toward the proximal end of the inner sheath 234. The length and material of the coil wire 238 may be adjusted to provide any desired contact pressure between the brush electrode 10 and the tissue being ablated. Further, the inner sheath 234 material and/or the embedded coil wire 238 may also be preshaped or prestressed to provide a desired curvature for efficient placement at particular tissue to be ablated.

As depicted in FIGS. 52 and 53, the inner sheath 234 defines a lumen 242 through which the conductor 48 extends. The uninsulated portion 52 of the conductor 48 is looped around the bundle of brush filaments 26 at a connection point 56 comprising part of the embedded portion 22 of the brush electrode 10. Other techniques, including but not limited to techniques discussed herein, may be used to transfer ablative energy from the conductor 48 to the brush electrode 10, including the mechanical connections depicted in, for example, FIGS. 24 and 25. If an outer sheath 236 is used in conjunction with the inner sheath 234 depicted in FIGS. 52 and 53, the outer sheath 236 may slide in the direction of arrow 244 along the outer surface of the inner sheath 234 to provide desired rigidity or bends, for example. By thus sliding the outer sheath 236 on the inner sheath 234, the separation distance between the distal end 246 of the outer sheath 236 and the distal end 240 of the inner sheath 234 may be adjusted. The outer sheath 236 may also be merely a lubricious skin to facilitate insertion of the catheter 232 to the ablation point. If desired, the embedded coil wire 238 may carry current.

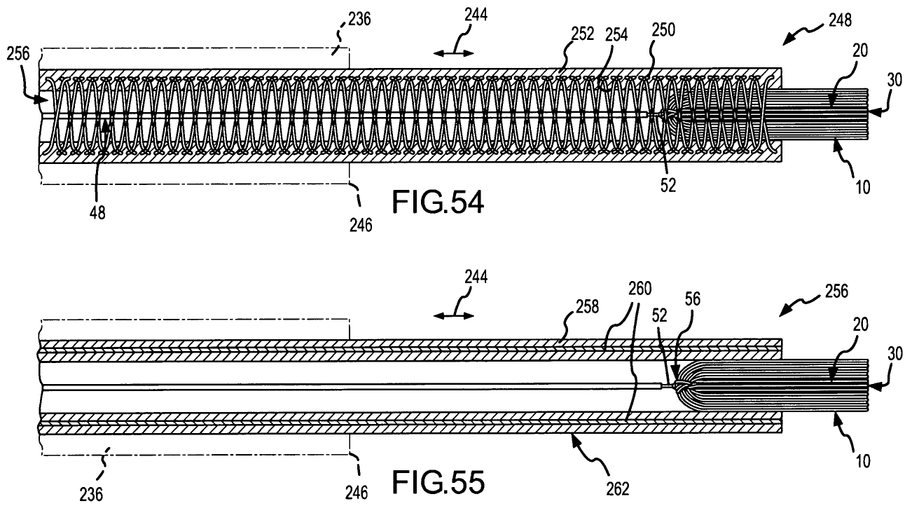

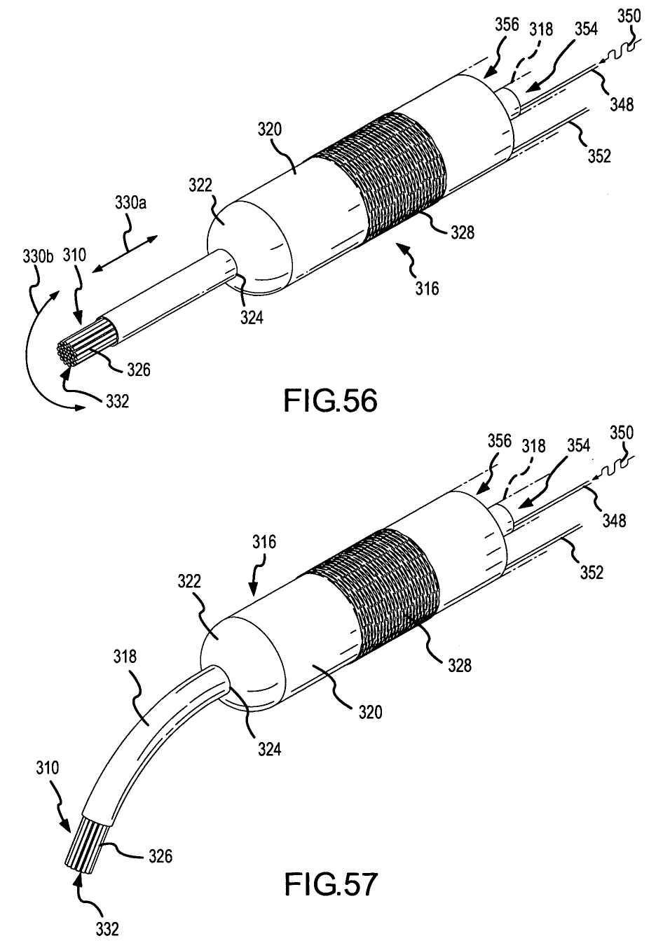

FIG. 54 is similar to FIG. 52, but is a fragmentary view in partial cross section of a braided wire sheath, brush electrode catheter 248 according to the present invention. In this embodiment, braided wire 250 is embedded in the material comprising the inner sheath 258. As with the other embodiments described above, the brush electrode 10 may be positioned longitudinally within the inner sheath 252 to set the length of the filaments 26 comprising the exposed portion 20 of the brush electrode 10. Once the brush electrode 10 is positioned at a desired longitudinal location within the inner sheath 252, it may be affixed to the inner sheath 252 to prevent the brush electrode 10 from moving relative to the inner sheath 252. Alternatively, the frictional engagement between the outer or circumferential surface of the bundle of filaments 26 comprising the brush electrode 10 and the inside surface 254 of the inner sheath 252 may be designed to provide sufficient, relative location stability between the brush electrode 10 and the inner sheath 252. Again, the inner sheath 252 defines a lumen 242 through which the conductor 48 may be routed to shield and protect the conductor 48 during placement of the brush electrode catheter 248 for ablation. The outer sheath 236 (shown in phantom in FIG. 54), if present, could be configured as described in connection with FIGS. 52 and 53. If desired, the braided wire 250 may be adapted to carry current.

FIG. 55 is a fragmentary view in partial cross section of a straight-wire sheath, brush electrode catheter 256 according to the present invention. The straight-wire sheath, brush electrode catheter 256 comprises a straight-wire inner sheath 258 that is similar to the coil-wire sheath 234 depicted in FIGS. 52 and 53, and to the braided-wire sheath 252 depicted in FIG. 54. In the straight-wire sheath 258, however, longitudinally-extending wires 260 are embedded in the material comprising the inner sheath 258. These longitudinally-extending wires 260 are radially offset from each other. The size, stiffness, and number of longitudinally-extending wires 260 are selected to give the distal portion 262 of the sheath 258 any desired pre-curve or rigidity. The outer sheath 236 (shown in phantom in FIG. 55), if present, is similar to the outer sheath shown and described in connection with FIGS. 5-54. The embedded, longitudinally-extending wires 260 may be current carrying.

The coil wire 238 depicted in FIGS. 52 and 53, the braided wire 250 depicted in FIG. 54, and the straight wires 260 depicted in FIG. 55 may be connected to a steering device (not shown) at the proximal end of the catheter to enable an operator (e.g., a physician) to actively direct or steer the working surface 30 of the brush electrode 10 toward tissue to be ablated.

FIGS. 56-58 depict an alternate embodiment of the invention in the form of a bipolar catheter 316 with a brush electrode 310. The bipolar catheter 316 is composed of an outer sheath 320 of a first diameter that substantially houses an inner flexible sheath 318 of a second, smaller diameter. The distal end of the outer sheath 320 may be formed as a rounded cap 322. The rounded cap 322 defines an opening or mouth 324 in the outer sheath 320 that provides a fluid seal around the outer surface of the flexible sheath 318. The distal end of the flexible sheath 318 extends distally from the mouth 324 of the outer sheath 320 and terminates with a brush electrode 310 composed of a plurality of filaments 326. The brush electrode 310 may be in the form of any of the embodiments of brush electrodes previously described herein. For example, the brush electrode 310, as depicted in FIGS. 56-58, may be of the type described with respect to FIGS. 2-5.

As shown in FIGS. 56 and 57, a dispersive return electrode 328 surrounds a portion of the outer surface of the outer sheath 320 at a distal end of the outer sheath 320 adjacent the rounded cap 322. The return electrode 328 may be formed of a biocompatible, conductive mesh, fabric, or braid wrapped about the outer sheath 320. The outer sheath 320 defines an outer lumen 356 that houses a return lead 352, which is electrically coupled with the dispersive return electrode 328 and also extends proximally the length of the outer sheath 320. The outer sheath 320 also houses the flexible sheath 318, which similarly extends proximally within the outer lumen 356 the length of the outer sheath 320. The flexible sheath 318 defines a fluid lumen 354 that, in addition to supplying conductive fluid to the brush electrode 310, also houses a conductor 348 for supplying RF energy 350 to the brush electrode 310 as previously described herein.

As depicted in FIG. 56, the distal end 332 of the brush electrode 310 may be moved proximally and distally, as well as radially from a longitudinal center of the outer sheath 320. The flexible sheath 318 may be moved proximally and distally (as indicated by the arrow 330a) by a clinician with respect to the outer sheath 320, thus sliding within the cap mouth 324 and correspondingly moving the distal tip 332 of the brush electrode 310. The flexible sheath 318 may also house one or more control wires (not shown) running the length of the flexible sheath 318 and anchored in the distal end of the flexible sheath 318 adjacent the brush electrode 310. When placed under tension, the control wire(s) cause the distal end of the flexible sheath 318 to bend in a lateral direction (as indicated by the arrow 330b). The distal tip 332 of the brush electrode 310 can be caused to deflect in any radial direction by rotation of the flexible sheath 318 by the clinician in conjunction with placing a control wire under tension. FIG. 57 depicts the distal end of the flexible sheath 318 and the attached brush electrode 310 in a deflected orientation.

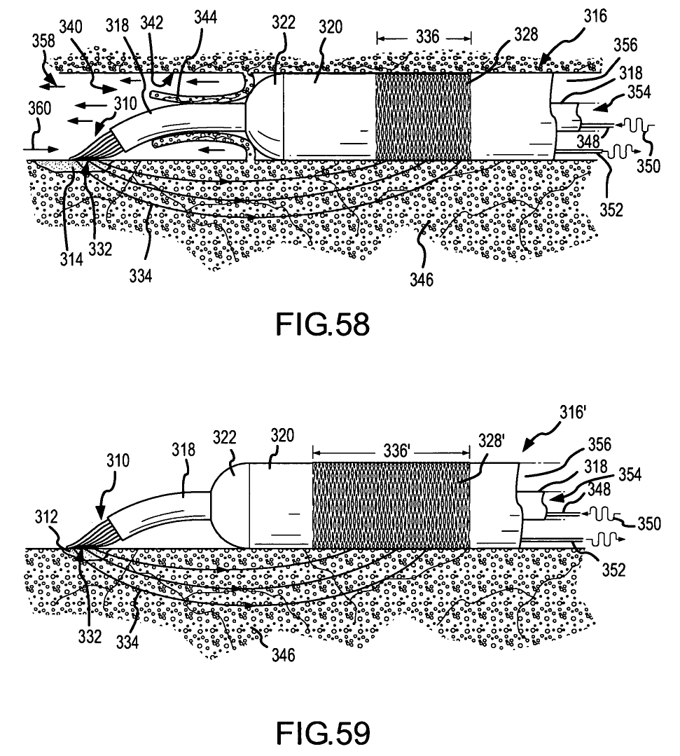

FIG. 58 depicts the bipolar catheter 316 previously described with respect to FIGS. 56 and 57 positioned within a vein 340, for example, a saphenous vein in the leg of a patient. The diameter of the outer sheath 320 of the bipolar catheter 316 is sized to ensure physical contact between the outer surface of the outer sheath 320, and more particularly the dispersive return electrode 328, and the interior veinal wall 342 of the vein 340. The flexible sheath 318 extends distally from the outer sheath 320, and happens to extend through a valve 344 in the vein 340 in the depiction of FIG. 58. The flexible sheath 318 is in a deflected orientation such that the distal tip 332 of the brush electrode 310 is in contact with the veinal wall 342.

The conductive filaments 326 of the brush electrode 310 are connected via the conductor 348 to an RF energy source (not shown). Conductive fluid from a fluid source (not shown) flows through the fluid lumen of the flexible sheath 318 and then interstitially through the filaments 326 to the distal tip 332 of the brush electrode 310. RF frequency current flows through the tissue 346 from the brush electrode 310 to the dispersive return electrode 328 on the outer sheath 320 as indicated by the electric field lines 334 in FIG. 58. The surface area of the dispersive return electrode 328, which is a function of the length 336 of the surface electrode and the diameter of the outer sheath 320, is designed to be greater than the contact area of the brush electrode 310 with the tissue 346. The current density (J) in the tissue 346 adjacent the brush electrode 310 is much higher than the current density at the return electrode 328. The rate of energy deposition (J²/σ, where σ is the electrical conductivity of the tissue 346) in the tissue 346 is, therefore, much greater adjacent the brush electrode 310 than at the dispersive return electrode 328. Consequently, lesions are formed in the tissue 346 only at the location of the brush electrode 310.

As indicated in FIG. 58 by the arrows 358, the blood in the vein 340 is flowing to the left of the page, while the catheter 316 is pulled in a retrograde direction to create a linear lesion 314 along the veinal wall 342 as indicated by the opposing arrow 360. Such a procedure may be desirable, for example, in the treatment of varicose veins to occlude and collapse a length of the vein 340. Generally, the occlusion of a saphenous vein using the bipolar brush electrode catheter 316 of the present invention requires the application of low power to the brush electrode 310 and a corresponding low fluid flow through the fluid lumen 354.

An alternative embodiment of a bipolar brush electrode catheter 316′ is depicted in FIG. 59. The structure of the catheter 316′ is substantially similar to the brush electrode catheter 316 depicted in FIGS. 56-58. As shown in FIG. 59, the catheter 316′ may be used in an open cavity or a vessel with a diameter much larger than the diameter of the outer sheath 320. The brush electrode 310 is being used to create a spot lesion 312 on the surface of a section of tissue 346. Thus, only a portion of the surface area of the dispersive return electrode 328′ may be in contact with the adjacent tissue 346, as shown in FIG. 59. As described above, the surface area of the dispersive return electrode 328′ in contact with the tissue 346 should be greater than the contact area of the brush electrode 310 with the tissue 346. Therefore, in applications in which only a portion of the dispersive return electrode 328′ may be in contact with the tissue 346, the size of the dispersive electrode 328′ should be increased accordingly to ensure that current density at the dispersive return electrode 328′ is much lower than at the brush electrode 310. For example, in order to achieve a surface area interface between the dispersive return electrode 328′ and the tissue 346 comparable to the surface area interface between the return electrode 328 and the tissue 346 in FIG. 58, the length 336′ of the dispersive return electrode 328′ of the catheter 316′ of FIG. 59 may be greater than the length 336 of the dispersive return electrode 328 of the catheter 316 of FIG. 58, presuming that the diameters of each of the catheters 316, 316′ is the same.

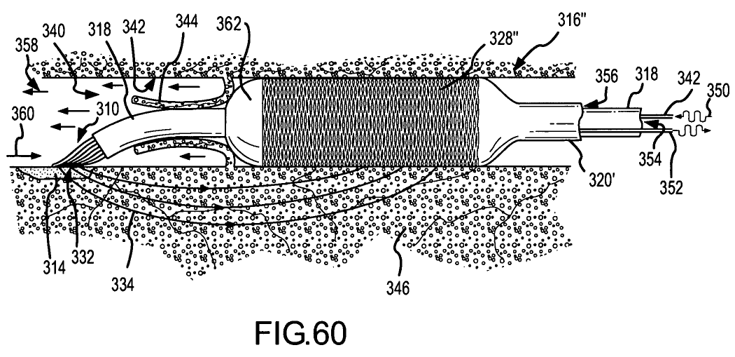

Yet another embodiment of a bipolar brush electrode catheter 316″ with a brush electrode 310 is depicted in FIG. 60. The bipolar catheter 316 is, as before, composed of an outer sheath 320′ of a first diameter that substantially houses an inner flexible sheath 318 of a second, smaller diameter. However, in this embodiment, the outer sheath 320′ is only slightly larger in diameter than the diameter of the flexible sheath. The distal end of the outer sheath 320′ comprises a dilatable balloon 362. The dilatable 362 may be attached to or a unitary portion of the outer sheath 320′. The dilatable balloon 362 is adapted to expand in diameter along a length of the flexible sheath 318 proximal to the brush electrode 310 in response to introduction and pressurization of a fluid. The distal end of the dilatable balloon 362 forms a fluid-tight seal around the outer surface of the flexible sheath 318. The distal end of the flexible sheath 318 extends distally from the dilatable balloon 362 and terminates with the brush electrode 310 composed of a plurality of filaments 326. The brush electrode 310 may be in the form of any of the embodiments of brush electrodes previously described herein.

As shown in FIG. 60, a dispersive return electrode 328″ surrounds a portion of the outer surface of the dilatable balloon 362. The return electrode 328″ may be formed of biocompatible, conductive materials including the following: mesh, fabric, polymer, or braid, wrapped about the dilatable balloon 362. Alternately, the dilatable balloon 362 may be coated with a thin conductive film or a vapor deposition of a biocompatible conductive metal, e.g., gold or platinum, which acts as the return electrode 328″. The film or vapor deposition may be solid or patterned. RF frequency current flows through the tissue 346 from the brush electrode 310 to the dispersive return electrode 328″ as indicated by the electric field lines 334 in FIG. 60. As with the previous embodiments of a bipolar electrode catheter, the surface area of the dispersive return electrode 328″, which is a function of the length of the dispersive return electrode 328″ and the diameter of the dilatable balloon 362 when extended, is designed to be greater than the contact area of the brush electrode 310 with the tissue 346.

The outer sheath 320′ defines an outer lumen 356 that houses a return lead 352, which is electrically coupled with the dispersive return electrode 328″ and also extends proximally the length of the outer sheath 320′. The outer lumen 356 is in fluid communication with the dilatable balloon 362. The outer lumen 356 transports fluid from a fluid source to the dilatable balloon 362 for expanding the dilatable balloon 362 to a sufficient diameter to place the return electrode 328″ on the outer surface of the dilatable electrode 362 in contact with adjacent tissue, for example, the interior wall of a vein as shown in FIG. 60. The outer sheath 320′ also houses the flexible sheath 318, which similarly extends proximally within the outer lumen 356 the length of the outer sheath 320′. The flexible sheath 318 defines a fluid lumen 354 that, in addition to supplying conductive fluid to the brush electrode 310, also houses a conductor 348 for supplying RF energy 350 to the brush electrode 310 as previously described herein.

As depicted in FIG. 60, the distal end 332 of the brush electrode 310 may be moved radially from a longitudinal center of the outer sheath 320′. The bipolar electrode catheter 316″ may be moved proximally and distally by a clinician (as indicated by the arrow 330a) within the vein 340. The flexible sheath 318 may also house one or more control wires (not shown) running the length of the flexible sheath 318 and anchored in the distal end of the flexible sheath 318 adjacent the brush electrode 310. When placed under tension, the control wire(s) cause the distal end of the flexible sheath 318 to bend in a lateral direction (as indicated by the arrow 330b). The distal tip 332 of the brush electrode 310 can thus be caused to deflect in any radial direction by rotation of the flexible sheath 318 by the clinician in conjunction with placing a control wire under tension.

FIG. 60 depicts the bipolar catheter 316″ previously described positioned within a vein 340, for example, a saphenous vein in the leg of a patient. The of the dilatable balloon 362 of the bipolar catheter 316″ is expanded to a diameter sized to ensure physical contact between the return electrode 328″ on the outer surface of the dilatable balloon 362 and the interior veinal wall 342 of the vein 340. The flexible sheath 318 extends distally from the dilatable balloon 362, and happens to extend through a valve 344 in the vein 340 in the depiction of FIG. 60. The flexible sheath 318 is in a deflected orientation such that the distal tip 332 of the brush electrode 310 is in contact with the veinal wall 342 to form a lesion 314.

In this embodiment, the catheter 316″ is again being used to create a spot lesion 312 on the surface of a section of tissue 346. In this application, the brush electrode 310 and distal end of the outer sheath 320 may be in an open cavity or a vessel with a diameter much larger than the diameter of the outer sheath 320. Thus, only a portion of the surface area of the dispersive return electrode 328′ may be in contact with the adjacent tissue 346, as shown in FIG. 59. As described above, the surface area of the dispersive return electrode 328′ in contact with the tissue 346 should be greater than the contact area of the brush electrode 310 with the tissue 346.

The catheter and brush electrode according to the present invention deliver ablative energy to the tissue via the conductive filaments alone, via the conductive fluid alone, or via both the conductive filaments and the conductive fluid. In the latter two configurations, the brush electrode is referred to as a wet-brush electrode. Since it is possible for the conductive fluid to escape from the exposed portion of the wet-brush electrode before reaching the working surface at the distal tip of the wet-brush electrode, there is some ablative energy leakage to the surrounding blood. The leakage of ablative energy to the surrounding blood is in part due to direct contact between the blood and the conductive filaments and in part due to the conductive fluid escaping between the filaments to the surrounding blood, particularly when substantial splaying of the filaments occurs (see, e.g., FIG. 49).

The design parameters for the brush electrode include both filament and brush parameters. The filament parameters include, for example, the material and structural properties of the individual filaments (e.g., what material(s) each individual filament is constructed from, whether the filaments are hollow or solid, whether the filaments are porous, and how flexible or stiff the filaments are), the shape and cross-sectional areas of the individual filaments, and the electrical conductivity of the individual filaments. The electrical conductivity of the individual filaments may be constant along the length of the filaments or may vary along the length of the filaments. Also, if the conductivity of a filament varies along its length, it may vary continuously or discontinuously. The filament design parameters may be different for each filament.

The design parameters for the brush electrode include, for example, the overall shape and cross-sectional area of the brush (i.e., the overall shape and size of the filament bundle forming the brush electrode), the tip length of the brush itself (i.e., the length of the portions of the filaments that extend the farthest from the distal end of the sheath surrounding the filament bundle), the shape of the brush tip, the length of the individual filaments relative to each other, the packing density of the filaments comprising the brush, and the overall electrical resistance of the brush. When both nonconductive and conductive filaments are present, the conductive filaments may be distributed evenly, randomly, or pseudo-randomly among the nonconductive filaments comprising the brush electrode.

By controlling, among other things, the cross-sectional shapes of the filaments, the cross-sectional areas of the filaments, the flexibility or stiffness of the filaments, the packing density of the filaments, the ratio of the nonconductive filaments to the conductive filaments, and the placement of the nonconductive and conductive filaments relative to each other, it is possible to obtain brush electrodes having desired electrical and thermal characteristics, which ultimately determine the types of lesions that may be obtained when using the brush electrodes for ablation. As mentioned above, it is even possible to vary the mechanical and electrical properties of each individual filament, if necessary, to achieve desired results.

The shapes and cross-sectional areas of the individual filaments and the packing density of the brush electrode affect the interstitial spaces between the filaments. The interstitial spaces between the filaments determine the flow path of the conductive or nonconductive fluid when the brush electrode is being used as a wet-brush electrode. The flow path of the conductive or nonconductive fluid determines to a great extent the electrical and thermal characteristics of the wet-brush electrode. The use of a large number of individual filaments defining interstitial spaces among the filaments results in efficient and effective cooling of the brush electrode and of the tissue surface. The effective cooling of the brush electrode achieved by the present invention reduces the formation of coagulum on the electrode, and the effective cooling of the tissue surface achieved by the present invention allows for the application of high-power RF energy for long durations, ultimately resulting in the formation of better lesions. Also, when using the brush electrode catheter in a blood-filled environment, the blood will further dilute the electric field created and act as a coolant, resulting in shallower lesions than would be created on “dry” tissue surfaces.

During use of a brush electrode as disclosed herein, the following operating parameters may be taken into account: the incidence angle between the brush electrode and the tissue, the stand-off distance between the brush electrode and the tissue, the power applied, the rate of fluid flow when present, and the duration of contact between the electrode and the tissue. Different effects (e.g., incision, ablation, coagulation, cauterization, fulguration, and desiccation) may be achieved with the brush electrode depending upon the presence or absence of fluid, the power of the therapeutic RF energy transmitted to the brush electrode, the waveform of the therapeutic energy, and the shape of the working surface of the brush electrode. For example, higher power RF energy is typically used for electrosurgical procedures such as creating incisions and cauterization. In addition, a sharply pointed working surface of the brush electrode may facilitate the creation of incisions, while a flatter working surface coupled with high energy may be preferable for large area cauterization. Alternately, lower power and a flatter working surface may be preferred for ablation or coagulation effects (e.g., for the treatment of varicose veins). However, a sharper working surface may be preferred for removal of tissue, for example, a tumor.

In one set of tests, Thunderon® filaments were used favorably in a wet-brush electrode having a circular cross section with an overall diameter of 6-8 French, a tip length of 2-3 millimeters, and electrical resistance of 100-150 ohms. In this embodiment, the size of the Thunderon® filaments was 40 decitex. When using this brush electrode with zero stand-off distance, 30 watts of power, saline flowing at 12 milliliters per minute, and contact between the wet-brush electrode and the tissue occurring for 60 seconds, 5-to-6 millimeter deep lesions were formed with an incidence angle of 90° between the wet-brush electrode and the tissue. Four millimeter deep lesions were formed when the incidence angle between the wet-brush electrode and the tissue was 0°. When a stand-off distance of 1 millimeter was used during tests with similar operating parameters, a slightly less deep (on the order of 3 millimeters deep) lesion was formed.

In another set of tests, lesions 3-13 millimeters deep were created using 20-50 watts of power and fluid flow rates of 3-18 milliliters per minute with wet-brush electrodes made from commercially available carbon fibers (e.g., carbon fibers available through Cytec Carbon Fibers LLC of South Carolina, United States of America). Isotonic saline infusion was used in these tests. (Isotonic saline is generally about twice as conductive as blood.) In other tests, linear lesions 20-42 millimeters long and 3-8 millimeters deep were created by applying 20-50 watts of power for 60 seconds in the presence of flow rates of 3-18 milliliters per minute using wet-brush electrodes produced with conductive filaments made from Thunderon®.

In a further set of experiments to simulate varicose vein treatment, in vitro tests on sheep veins were performed. A 6 Fr (Fr=French=⅓ mm) wet-brush electrode occluded 20 mm length areas of sheep veins using 10 W of power, a fluid flow rate of 1 ml/min, and a period of 120 seconds. In contrast to prior art metal electrodes for varicose vein treatment, there was no coagulum formation on the brush electrode during the ablation procedure and no charring of the veins was detected. Further, the wet-brush electrode did not stick to the target tissue as is often the case during varicose vein treatment using prior art metal electrodes.

In another experiment, a bipolar form of the invention was used to form lesions in tissue. A 6 Fr wet-brush electrode catheter was inserted in a 12 Fr sheath. A 9 mm wide annular dispersive return electrode was mounted circumferentially about the 12 Fr sheath. The catheter and sheath combination were inserted between two slabs of bovine heart tissue. Spot lesions in the tissue of 3-4 mm deep were observed after application of energy for 60 seconds at 5 W and a fluid flow rate of 1 ml/min.

As already mentioned, when conductive fluid is used, the brush electrode becomes a wet-brush electrode. In a wet-brush electrode, the conductive fluid serves both thermodynamic functions and electrical functions. Thermodynamically, the conductive fluid cools both the electrode and the tissue surface. As previously mentioned, effective cooling of the electrode inhibits or prevents coagulum formation on the electrode in blood-filled environments; and effective cooling of the tissue surface permits longer application of relatively high RF energy, resulting in the formation of the deeper lesions. Electrically, the conductive fluid serves as a virtual electrode. The conductive fluid also insulates the conductive brush filaments from the surrounding blood in blood-filled environments, which helps prevent the formation of coagulum.

The conductive fluid also creates a conductivity gradient resulting from a concentration gradient. The conductive fluid flowing interstitially through the brush filaments has a field homogenizing effect. This results in the formation of a homogeneous lesion in the tissue when the tissue is of the same type. The conductive fluid flowing through the working surface at the distal tip of the wet-brush electrode thus helps to mitigate hot spots resulting from edge effects. Further, since the number of edges present in a brush electrode greatly exceeds the number of edges present in many existing electrodes, the energy build up at each filament edge in a brush electrode is less than it would be for existing electrodes, assuming the same power setting. This results in less severe edge effects when using the brush electrode of the present invention. The conductive fluid, when used, further smoothes or reduces the undesirable edge effects.

In the present invention, the filaments of the wet-brush electrode serve both mechanical and electrical functions. Mechanically, the filaments create a flexible electrode that provides improved tissue contact. The filaments also create interstitial spaces, which not only provides effective fluid channeling, but also prevents the “virtual electrode” from being washed away by the surrounding blood in blood-filled environments, and helps to smooth the concentration gradient of the conductive fluid. Electrically, the filaments serve as a conductive electrode.

Again, it should be noted that although the filaments are depicted in nearly all of the figures as having circular cross sections for simplicity, the individual filaments may intentionally or unintentionally have a wide variety of cross-sectional configurations and areas, and need not be circular. Manufacturing irregularities may result in various cross-sectional configurations, or filaments having a variety of different cross-sectional configurations may be intentionally selected to achieve a desired electric field at the brush-tissue interface. The number of filaments in the bundles of filaments of the brush electrodes depicted in the figures herein are meant to be representative only and are reflective of the limitations of line drawings. It should be recognized that a bundle of filaments may be composed of hundreds, thousands, or (in the case of carbon fibers, for example) tens of thousands of individual filaments. This provides an enormous increase in the surface area contact between the brush electrode and the tissue as compared to prior electrodes resulting in faster and improved energy transfer to the tissue. Reduction in the time of electrode-tissue contact reduces heat generated and thereby reduces the risk of tissue charring. The filaments also may not be perfectly aligned longitudinally. Further, the filaments may comprise a yarn of braided or twisted groups of fibers, or the filaments may comprise a roving pattern of untwisted, longitudinally-extending, substantially-parallel, conductive and nonconductive fibers.

Although various embodiments of this invention have been described above with a certain degree of particularity, or with reference to one or more individual embodiments, those skilled in the art could make numerous alterations to the disclosed embodiments without departing from the spirit or scope of this invention. It is intended that all matter contained in the above description and shown in the accompanying drawings shall be interpreted as illustrative only of particular embodiments and not limiting. All directional references (e.g., proximal, distal, upper, lower, upward, downward, left, right, lateral, front, back, top, bottom, above, below, vertical, horizontal, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the basic elements of the invention as defined in the following claims.

1. A bipolar electrode catheter comprising
a flexible sheath with a distal end;
a conforming electrode adapted to apply therapeutic energy to target tissue, said conforming electrode comprising an embedded portion and an exposed portion,
wherein said embedded portion extends into said distal end of said flexible sheath,
wherein said exposed portion has a distal end that forms a working surface, and
wherein said exposed portion extends from said distal end of said flexible sheath;
a conductor electrically coupled with said conforming electrode and adapted to carry therapeutic energy from an energy source to said conforming electrode;
a return electrode positioned about said flexible sheath proximal to said conforming electrode; and
a return lead electrically coupled with said return electrode.

2. The bipolar electrode catheter of claim 1, further comprising an outer sheath surrounding said flexible sheath, wherein
said distal end of said flexible sheath protrudes distally from a distal end of said outer sheath; and
said return electrode is positioned about an outer surface of said outer sheath.

3. The bipolar electrode catheter of claim 2, wherein
said outer sheath defines an outer lumen; and
said flexible sheath is moveably mounted within and adapted to slide longitudinally with respect to said outer lumen;
whereby a separation distance between said distal end of said outer sheath and said distal end of said flexible sheath is adjustable.

4. The bipolar electrode catheter of claim 1, further comprising
an outer sheath surrounding said flexible sheath; and
a dilatable balloon joined with a distal end of said outer sheath and extending distally therefrom about said flexible sheath; wherein
said distal end of said flexible sheath protrudes distally from a distal end of said dilatable balloon;
said distal end of said dilatable balloon seals against an outer surface of said flexible sheath; and
said return electrode is positioned about an outer surface of said dilatable balloon.

5. The bipolar electrode catheter of claim 4, wherein said outer surface of said dilatable balloon is said return electrode.

6. The bipolar electrode catheter of claim 4, wherein said outer sheath further defines a lumen that delivers fluid from a fluid source to said dilatable balloon.

7. The bipolar electrode catheter of claim 1, wherein said flexible sheath further defines a lumen that delivers fluid from a fluid source to said conforming electrode.

8. The bipolar electrode catheter of claim 1, wherein
said working surface of said conforming electrode defines a first surface area;
said return electrode defines a second surface area; and
said second surface area is greater than said first surface area.

9. The bipolar electrode catheter of claim 1 further comprising an actuation mechanism for deflecting said distal end of said flexible catheter.

10. The bipolar electrode catheter of claim 1, wherein the conforming electrode further comprises
a plurality of flexible filaments that directly or indirectly transfer the therapeutic energy to the target tissue.

11. The bipolar electrode catheter of claim 10, further comprising an outer sheath surrounding said flexible sheath, wherein
said distal end of said flexible sheath protrudes distally from a distal end of said outer sheath; and
said return electrode is positioned about an outer surface of said outer sheath.

12. The bipolar electrode catheter of claim 11, wherein
said outer sheath defines an outer lumen; and
said flexible sheath is moveably mounted within and adapted to slide longitudinally with respect to said outer lumen;
whereby a separation distance between said distal end of said outer sheath and said distal end of said flexible sheath is adjustable.

13. The bipolar electrode catheter of claim 10, wherein
each of said plurality of flexible filaments defines a distal tip;
said distal tips together form a working surface;
said working surface defines a first surface area;
said return electrode defines a second surface area; and
said second surface area is greater than said first surface area.

14. The bipolar electrode catheter of claim 10, wherein
said flexible sheath further defines a lumen that delivers fluid from a fluid source to said plurality of flexible filaments;
each of said plurality of flexible filaments has a longitudinal axis and is aligned generally parallel to others of said plurality of flexible filaments with respect to said longitudinal axis;
said plurality of flexible filaments define interstitial spaces among said plurality of flexible filaments; and
said interstitial spaces direct said fluid predominantly parallel to said longitudinal axes of said plurality of flexible filaments.

15. The bipolar electrode catheter of claim 14, wherein said fluid is a conductive fluid.

16. The bipolar electrode catheter of claim 10, wherein
each of said plurality of flexible filaments defines a distal tip;
said distal tips are trimmed to create a shaped working surface; and
said shaped working surface is selected from a group consisting of a relatively flat surface, a blade, a point, a cone, a trough, an arch, a dome, bowl, and a channel.

17. The bipolar electrode catheter of claim 10, wherein said plurality of flexible filaments is arranged in a bundle that is folded and inserted at least partially into a lumen in said distal end of said flexible sheath.

18. The bipolar electrode catheter of claim 10, wherein at least some of said plurality of flexible filaments comprise tapered filaments.

19. The bipolar electrode catheter of claim 18, wherein
each of said plurality of flexible filaments defines a distal tip;
at said distal end of said flexible sheath, said tapered filaments have larger cross-sectional areas than said tapered filaments have at said distal tips of said tapered filaments.

20. The bipolar electrode catheter of claim 10, wherein at least a first portion of said plurality of flexible filaments comprises a conductive material.

21. The bipolar electrode catheter of claim 20, wherein each flexible filament in said first portion of said plurality of flexible filaments has a longitudinal axis, and wherein each flexible filament in said first portion has varying conductivity along said longitudinal axis.

22. The bipolar electrode catheter of claim 20, wherein
at least a second portion of said plurality of flexible filaments comprises a nonconductive material, and
each flexible filament in said second portion is longer than each filament in said first portion.

23. The bipolar electrode catheter of claim 20, wherein
each of said plurality of flexible filaments defines a distal tip;
said distal tips of said first portion of said plurality of flexible filaments are nonconductive tips.

24. The bipolar electrode catheter of claim 10, wherein said plurality of flexible filaments are selected from the group consisting of acrylic fibers, metal fibers, metal plated fibers, conductively-coated fibers, carbon fibers, and carbon-compound fibers.

25. The bipolar electrode catheter of claim 10, wherein at least a portion of said plurality of flexible filaments comprises hollow filaments.

26. The bipolar electrode catheter of claim 10, wherein at least a portion of said plurality of flexible filaments comprises porous filaments.

27. The bipolar electrode catheter of claim 10, wherein said plurality of flexible filaments further comprises a lead, wherein a distal end of said lead is embedded within said plurality of flexible filaments.

28. The bipolar electrode catheter of claim 27, further comprising a device operatively coupled with said lead.

29. The bipolar electrode catheter of claim 28, wherein said device is selected from a group consisting of a thermal sensor, a pressure sensor, and an ultrasound sensor.

30. The bipolar electrode catheter of claim 10, further comprising an energy transfer coil, wherein
a proximal portion of said plurality of filaments comprises said embedded portion;
said energy transfer coil surrounds at least a portion of said embedded portion of said plurality of filaments; and
said conductor is electrically coupled with said energy transfer coil to transfer said therapeutic energy to said plurality of flexible filaments.

31. The bipolar electrode catheter of claim 10 further comprising an energy transfer mesh, wherein
a proximal portion of said plurality of filaments comprises said embedded portion;
said energy transfer mesh surrounds at least a portion of said embedded portion of said plurality of filaments; and
said conductor is electrically coupled with said energy transfer mesh to transfer said therapeutic energy to said plurality of flexible filaments.

32. The bipolar electrode catheter of claim 14, wherein
a proximal portion of said plurality of filaments comprises said embedded portion and a distal portion of said plurality of filaments comprises said exposed portion;
said flexible sheath further comprises a plurality of tubes arranged in a circumferential ring around said lumen;
said embedded portion resides in said lumen at said distal end of said flexible sheath; and
said plurality of tubes transport said fluid to said exposed portion of said brush electrode.

33. The bipolar electrode catheter of claim 14 further comprising
a porous inner sheath residing within said lumen, defining an inner lumen, and extending to said distal end of said flexible catheter, and wherein
a proximal portion of said plurality of filaments comprises said embedded portion and a distal portion of said plurality of filaments comprises said exposed portion;
said embedded portion resides in said inner lumen at said distal end of said flexible catheter; and
said porous inner sheath transports said fluid to said exposed portion of said plurality of flexible filaments.

34. The bipolar electrode catheter of claim 14, wherein
said flexible sheath comprises a threaded outer surface at said distal end;
a proximal portion of said plurality of filaments comprises said embedded portion and a distal portion of said plurality of filaments comprises said exposed portion;
said embedded portion resides within said lumen at said distal end of said flexible sheath; and wherein
said bipolar electrode further comprises a cover surrounding said threaded outer surface of said flexible sheath,
whereby a helical flow channel is defined between said threaded outer surface and said cover and transports fluid to said exposed portion of said plurality of flexible filaments.

35. The bipolar electrode catheter of claim 14, wherein
said flexible sheath comprises a grooved outer surface including at least one longitudinally-extending groove;
a proximal portion of said plurality of filaments comprises said embedded portion and a distal portion of said plurality of filaments comprises said exposed portion;
said embedded portion resides within said lumen at said distal end of said shaft; and wherein
said bipolar electrode catheter further comprises a cover surrounding said grooved outer surface of said flexible sheath,
whereby at least one longitudinally-extending flow channel is defined between said grooved outer surface and said cover and transports fluid to said exposed portion of said plurality of flexible filaments.

36. A bipolar surgical device for transferring therapeutic energy to tissue, the bipolar surgical device comprising
a flexible sheath having a distal end and defining a fluid lumen;
a brush electrode through which said therapeutic energy is applied to target tissue, said brush electrode mounted at said distal end of said flexible sheath and further comprising
an embedded portion; and
an exposed portion; wherein
said embedded portion is in fluid communication with said fluid lumen,
said exposed portion has a distal end forming a working surface, and
said exposed portion extends from said distal end of said flexible sheath; and
a conductor electrically coupled with said brush electrode and adapted to carry said therapeutic energy from an energy source to said brush electrode;
an outer sheath surrounding said flexible sheath and defining an outer lumen, wherein
said distal end of said flexible sheath protrudes distally from a distal end of said outer sheath; and
said flexible sheath is moveably mounted within and adapted to slide longitudinally with respect to said outer lumen;
whereby a separation distance between said distal end of said outer sheath and said distal end of said flexible sheath is adjustable; and
a return electrode positioned about an outer surface of said outer sheath;
a return lead electrically coupled with said return electrode.

37. The bipolar surgical device of claim 36, wherein said brush electrode comprises a plurality of filaments having interstitial gaps between said filaments and said interstitial gaps direct the fluid toward said working surface.

38. The bipolar surgical device of claim 36 further comprising a mechanical interface for making electrical contact between said embedded portion of said brush electrode and said conductor.

39. The bipolar surgical device of claim 38, wherein
a distal end of said fluid carrying lumen is bounded by said mechanical interface; and
said mechanical interface is porous.

40. The bipolar surgical device of claim 36, wherein
said working surface of said brush electrode defines a first surface area;
said return electrode defines a second surface area; and
said second surface area is greater than said first surface area.

41. The bipolar surgical device of claim 36 further comprising an actuation mechanism for deflecting said distal end of said flexible catheter.

42. A method for treating varicose veins, the method comprising
inserting a bipolar electrode catheter into a lumen of a varicose vein;
pulling said bipolar electrode catheter in a retrograde direction within the lumen of the varicose vein;
maintaining contact between an interior wall of the varicose vein and an annular dispersive return electrode positioned about said bipolar electrode catheter proximal to a distal end of said bipolar electrode catheter;
dragging a brush electrode mounted at said distal end of said bipolar electrode catheter along a length of the interior wall of the varicose vein while pulling said bipolar electrode catheter;
energizing said brush electrode with therapeutic energy from an energy source while dragging said brush electrode;
transferring said therapeutic energy from said brush electrode to the interior wall of the varicose vein to occlude the varicose vein; and
returning said therapeutic energy from the varicose vein to said bipolar electrode catheter at said annular dispersive return electrode.

43. The method of claim 42 further comprising
applying conductive fluid to interior wall of the varicose vein via said brush electrode during the step of energizing.

44. The method of claim 42, wherein the step of maintaining contact further comprises dilating a balloon positioned about said bipolar electrode catheter proximal to a distal end of said bipolar electrode catheter, wherein said dispersive return electrode covers at least a portion of an outer surface of said balloon.
